(19) United States
(12) Patent Application Publication
(10) Pub. No.: US 2026/0265227 A1
(43) Pub. Date: Sep. 10, 2026

(54) AMIDE PRODRUGS AND USES THEREOF

(71) Applicant: Autobahn Therapeutics, Inc., San Diego, CA (US)

(72) Inventors: Jason Randall HARRIS, San Diego, CA (US); Jill Melissa BACCEI, San Diego, CA (US); Brian Andrew STEARNS, San Diego, CA (US)

(21) Appl. No.: 18/880,174

(22) PCT Filed: Jun. 30, 2023
(86) PCT No.: PCT/US2023/069491

§ 371 Date: Dec. 30, 2024

Related U.S. Application Data
(60) Provisional application No. 63/367,569, filed on Jul. 1, 2022.

(51) Int. Cl. C07D 403/12; A61K 31/397; A61K 31/4427; A61K 31/455; A61K 31/501; A61K 31/506; C07D 205/04; C07D 401/12

(52) U.S. Cl. CPC C07D 403/12; A61K 31/397; A61K 31/4427; A61K 31/455; A61K 31/501; A61K 31/506; C07D 205/04; C07D 401/12

(57) ABSTRACT

Described herein are fatty acid amide hydrolase (FAAH) cleavable prodrugs of compounds that modulate sphingosine-1-phosphate receptor 5 (S1P₅), methods of making such compounds, pharmaceutical compositions and medicaments comprising such compounds and methods of using such compounds in treating a CNS disease or disorder.

### CROSS-REFERENCE

This application claims benefit of U.S. Provisional Patent Application No. 63/367,569, filed on Jul. 1, 2022 which is incorporated herein by reference in its entirety.

### BACKGROUND OF THE INVENTION

The blood-brain barrier is composed of tightly linked endothelial cells that limit the passage of pathogens and specific types of small and large molecules from the blood into the brain. This critical protective function also restricts the diffusion of therapeutics into the brain representing a major challenge to the development of new medicines for CNS diseases.

### SUMMARY OF THE INVENTION

In one aspect, described herein is a compound that has the structure of Formula (I), or a pharmaceutically acceptable salt or solvate thereof:

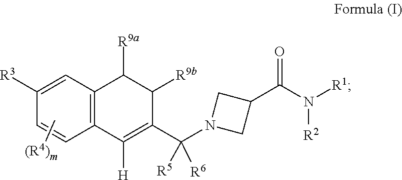

wherein:
R¹ is selected from hydrogen, —OR⁷, —NR⁷R⁸, C₁-C₆alkyl, C₂-C₆alkenyl, C₂-C₆alkynyl, C₃-C₆cycloalkyl, C₂-C₆heterocycloalkyl, C₁₋₉heteroaryl, phenyl, and —C₁-C₆alkyl-phenyl, wherein C₁-C₆alkyl, C₂-C₆alkenyl, C₂-C₆alkynyl, C₃-C₆cycloalkyl, C₂-C₉heterocycloalkyl, C₁₋₉heteroaryl, phenyl, and —C₁-C₆alkyl-phenyl are optionally substituted with one or more groups selected from halogen, C₁-C₆alkyl, C₁₋₆ haloalkyl, C₃-C₆cycloalkyl, —CN, —OR¹⁰, —N(R¹⁰)(R¹¹), and —S(O)₂R¹³;
R² is selected from hydrogen, —OR⁷, —NR⁷R⁸, C₁-C₆alkyl, C₂-C₆alkenyl, C₂-C₆alkynyl, C₃-C₆cycloalkyl, C₂-C₆heterocycloalkyl, C₁₋₉heteroaryl, phenyl, and —C₁-C₆alkyl-phenyl, wherein C₁-C₆alkyl, C₂-C₆alkenyl, C₂-C₆alkynyl, C₃-C₆cycloalkyl, C₂-C₉heterocycloalkyl, C₁₋₉heteroaryl, phenyl, and —C₁-C₆alkyl-phenyl are optionally substituted with one or more groups selected from halogen, C₁-C₆alkyl, C₁₋₆ haloalkyl, C₃-C₆cycloalkyl, —CN, —OR¹⁰, —N(R¹⁰)(R¹¹), and —S(O)₂R¹³; or R¹ and R² are combined to form a C₂-C₉heterocycloalkyl optionally substituted with one or more groups selected from halogen, C₁₋₆alkyl, C₁₋₆haloalkyl, —OR¹⁰, —N(R¹⁰)(R¹¹), and —S(O)₂R¹³;
R³ is selected from halogen, —CN, C₁₋₆alkyl, C₁₋₆haloalkyl, C₂₋₆alkenyl, C₂₋₆alkynyl, —OR¹⁰, —SR¹⁰, —N(R¹⁰)(R¹¹), —C(O)R¹³, —S(O)₂R¹³, and —S(O)₂N(R¹⁰)(R¹¹)—, wherein C₁₋₆ alkyl, C₂₋₆alkenyl, and C₂₋₆alkynyl, are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R⁴ is independently selected from halogen, —CN, C₁₋₆alkyl, C₁₋₆haloalkyl, C₂₋₆ alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²) C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
R⁵ and R⁶ are independently selected from hydrogen and C₁-C₆alkyl;
each R⁷ and each R⁸ are independently selected from hydrogen and C₁-C₆alkyl;
R^{9a} and R^{9b} are independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), wherein C₁₋₆alkyl, C₂₋₆alkenyl, C₂₋₆ alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, —C₁₋₆alkyl-C₃₋₆cycloalkyl, C₂₋₉heterocycloalkyl, —C₁₋₆ alkyl-C₂₋₉heterocycloalkyl, C₆₋₁₀aryl, —C₁₋₆alkyl-C₆₋₁₀aryl, C₁₋₉heteroaryl, and —C₁₋₆ alkyl-C₁₋₉heteroaryl, wherein C₁₋₆alkyl, C₂₋₆alkenyl, C₂₋₆alkynyl, C₃₋₆cycloalkyl, —C₁₋₆ alkyl-C₃₋₆cycloalkyl, C₂₋₉heterocycloalkyl, —C₁₋₆alkyl-C₂₋₉heterocycloalkyl, C₆₋₁₀aryl, —C₁₋₆alkyl-C₆₋₁₀aryl, C₁₋₉heteroaryl, and —C₁₋₆alkyl-C₁₋₉heteroaryl are optionally substituted with one, two, or three groups selected from halogen, —CN, hydroxy, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉ heterocycloalkyl, C₆₋₁₀aryl, and C₁₋₉heteroaryl, wherein C₁₋₆alkyl, C₂₋₆alkenyl, C₂₋₆ alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
m is 0, 1, 2, or 3.

In some embodiments is a compound of Formula (I), or a pharmaceutically acceptable salt or solvate thereof, wherein R⁵ and R⁶ are hydrogen. In some embodiments is a compound of Formula (I), or a pharmaceutically acceptable salt or solvate thereof, wherein R^{9a} and R^{9b} are independently selected from hydrogen and C₁₋₆alkyl. In some embodiments is a compound of Formula (I), or a pharmaceutically acceptable salt or solvate thereof, wherein R^{9a} is hydrogen and R^{9b} is C₁₋₆alkyl. In some embodiments is a compound of Formula (I), or a pharmaceutically acceptable salt or solvate thereof, wherein R^{9a} is hydrogen and R^{9b} is —CH₃. In some embodiments is a compound of Formula (I), or a pharmaceutically acceptable salt or solvate thereof, wherein R³ is selected from C₁₋₆alkyl, C₁₋₆haloalkyl, —OR¹⁰, —SR¹⁰, —N(R¹⁰)(R¹¹), and —C(O)R¹³, wherein C₁₋₆alkyl is optionally substituted with one, two, or three groups selected from halogen, C₁₋₆haloalkyl, —OR¹⁰, and —N(R¹⁰)(R¹¹). In some embodiments is a compound of Formula (I), or a pharmaceutically acceptable salt or solvate thereof, wherein R³ is selected from C₁₋₆alkyl, C₁₋₆haloalkyl, —OR¹⁰, —SR¹⁰, and —N(R¹⁰)(R¹¹), wherein C₁₋₆alkyl is optionally substituted with one group selected from —OR¹⁰ and —N(R¹⁰)(R¹¹). In some embodiments is a compound of Formula (I), or a pharmaceutically acceptable salt or solvate thereof, wherein R³ is selected from —OR¹⁰, —SR¹⁰, and —N(R¹⁰)(R¹¹), wherein R¹⁰ and R¹¹ are independently selected from hydrogen, C₁₋₆alkyl, and C₁₋₆haloalkyl. In some embodiments is a compound of Formula (I), or a pharmaceutically acceptable salt or solvate thereof, wherein R³ is selected from —O(CH₂)₃CF³, —O(CH₂)₄CF³, —O(CH₂)₅CF³, —S(CH₂)₃CF³, —S(CH₂)₄CF³, —S(CH₂)₅CF³, —N(H)(CH₂)₃CF³, —N(H)(CH₂)₄CF³, and —N(H)(CH₂)₅CF³. In some embodiments is a compound of Formula (I), or a pharmaceutically acceptable salt or solvate thereof, wherein m is 0.

In some embodiments is a compound of Formula (I), or a pharmaceutically acceptable salt or solvate thereof, having the structure of Formula (Ia):

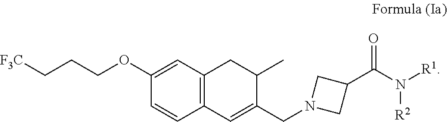

In some embodiments is a compound of Formula (I), or a pharmaceutically acceptable salt or solvate thereof, having the structure of Formula (Iaa):

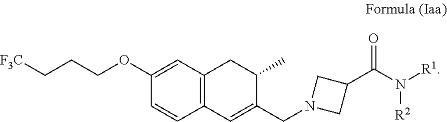

In some embodiments is a compound of Formula (I), (Ia), or (Iaa), or a pharmaceutically acceptable salt or solvate thereof, wherein R¹ is hydrogen. In some embodiments is a compound of Formula (I), (Ia), or (Iaa), or a pharmaceutically acceptable salt or solvate thereof, wherein R¹ is C₁-C₆alkyl. In some embodiments is a compound of Formula (I), (Ia), or (Iaa), or a pharmaceutically acceptable salt or solvate thereof, wherein R² is C₁-C₆alkyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (I), (Ia), or (Iaa), or a pharmaceutically acceptable salt or solvate thereof, wherein R² is C₁-C₆alkyl substituted with one or more groups selected from halogen, C₁₋₆haloalkyl, —OR¹⁰, and —N(R¹⁰)(R¹¹). In some embodiments is a compound of Formula (I), (Ia), or (Iaa), or a pharmaceutically acceptable salt or solvate thereof, wherein R² is C₁-C₆alkyl substituted with one or more-OH. In some embodiments is a compound of Formula (I), (Ia), or (Iaa), or a pharmaceutically acceptable salt or solvate thereof, wherein R² is C₁-C₆alkyl substituted with one or more of halogen. In some embodiments is a compound of Formula (I), (Ia), or (Iaa), or a pharmaceutically acceptable salt or solvate thereof, wherein R² is unsubstituted C₁-C₆alkyl. In some embodiments is a compound of Formula (I), (Ia), or (Iaa), or a pharmaceutically acceptable salt or solvate thereof, wherein R² is —CH₃. In some embodiments is a compound of Formula (I), (Ia), or (Iaa), or a pharmaceutically acceptable salt or solvate thereof, wherein R² is C₃-C₆cycloalkyl optionally substituted with one or more groups selected from halogen, C₁-C₆alkyl, C₁₋₆haloalkyl, —OR¹⁰, and —N(R¹⁰)(R¹¹). In some embodiments is a compound of Formula (I), (Ia), or (Iaa), or a pharmaceutically acceptable salt or solvate thereof, wherein R² is unsubstituted C₃-C₆cycloalkyl. In some embodiments is a compound of Formula (I), (Ia), or (Iaa), or a pharmaceutically acceptable salt or solvate thereof, wherein R² is C₁₋₉heteroaryl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (I), (Ia), or (Iaa), or a pharmaceutically acceptable salt or solvate thereof, wherein R² is C₁₋₉ heteroaryl optionally substituted with one or more groups selected from halogen, C₁-C₆alkyl, C₁₋₆haloalkyl, —CN, —OR¹⁰, —N(R¹⁰)(R¹¹), and —S(O)₂R¹³, wherein C₁₋₉heteroaryl is selected from pyridyl, pyridazinyl, pyrimidinyl, and pyrazinyl. In some embodiments is a compound of Formula (I), (Ia), or (Iaa), or a pharmaceutically acceptable salt or solvate thereof, wherein R² is selected from unsubstituted pyridyl, unsubstituted pyridazinyl, unsubstituted pyrimidinyl, and unsubstituted pyrazinyl. In some embodiments is a compound of Formula (I), (Ia), or (Iaa), or a pharmaceutically acceptable salt or solvate thereof, wherein R² is hydrogen. In some embodiments is a compound of Formula (I), (Ia), or (Iaa), or a pharmaceutically acceptable salt or solvate thereof, wherein R¹ and R² are combined to form a C₂-C₉heterocycloalkyl optionally substituted with one or more groups selected from halogen, C₁₋₆alkyl, C₁₋₆haloalkyl, —OR¹⁰, —N(R¹⁰)(R¹¹), and —S(O)₂R¹³. In some embodiments is a compound of Formula (I), (Ia), or (Iaa), or a pharmaceutically acceptable salt or solvate thereof, wherein R¹ and R² are combined to form an unsubstituted C₂-C₆heterocycloalkyl.

In another aspect, described herein is a pharmaceutical composition comprising a compound of Formula (I), (Ia), or (Iaa), or a pharmaceutically acceptable salt or solvate thereof, and at least one pharmaceutically acceptable excipient. In some embodiments is a pharmaceutical composition comprising a compound of Formula (I), (Ia), or (Iaa), or a pharmaceutically acceptable salt or solvate thereof, and at least one pharmaceutically acceptable excipient, further comprising a peripherally restricted fatty acid amide hydrolase (FAAH) inhibitor. In some embodiments is a pharmaceutical composition comprising a compound of Formula (I), (Ia), or (Iaa), or a pharmaceutically acceptable salt or solvate thereof, and at least one pharmaceutically acceptable excipient, further comprising a peripherally restricted fatty acid amide hydrolase (FAAH) inhibitor, wherein the peripherally restricted FAAH inhibitor is ASP-3652.

In another aspect, described herein is a method of treating a CNS disease or disorder in a patient in need thereof comprising administering to the patient a compound of Formula (I), (Ia), or (Iaa), or a pharmaceutically acceptable salt or solvate thereof. In some embodiments is a method of treating a CNS disease or disorder in a patient in need thereof comprising administering to the patient a compound of Formula (I), (Ia), or (Iaa), or a pharmaceutically acceptable salt or solvate thereof, wherein the CNS disease or disorder is selected from acute disseminated encephalomyelitis (ADEM), acute hemorrhagic leukoencephalitis (AHL or AHLE), adult Refsum disease, infantile Refsum disease, Alexander disease, Alzheimer's disease, Balo concentric sclerosis, Canavan disease, central pontine myelinolysis (CPM), cerebral palsy, cerebrotendineous xanthomatosis, chronic inflammatory demyelinating polyneuropathy (CIDP), Devic's syndrome, diffuse myelinoclastic sclerosis, encephalomyelitis, Guillain-Barre syndrome, Huntington's disease (HD), idiopathic inflammatory demyelinating disease (HDD), Krabbe disease, Leber hereditary optic neuropathy, leukodystrophy, Marburg multiple sclerosis, Marchiafava-Bignami disease, metachromatic leukodystrophy (MLD), multifocal motor neuropathy (MMN), multiple sclerosis (MS), Neiman Pick Type-C, paraproteinemic demyelinating polyneuropathy, Parkinson's disease (PD), Pelizaeus-Merzbacher disease (PMD), progressive multifocal leukoencephalopathy (PML), tropical spastic paraparesis (TSP), X-linked adrenoleukodystrophy (X-ALD, ALO, or X-linked ALO), and Zellweger syndrome. In some embodiments is a method of treating a CNS disease or disorder in a patient in need thereof comprising administering to the patient a compound of Formula (I), (Ia), or (Iaa), or a pharmaceutically acceptable salt or solvate thereof, wherein the CNS disease or disorder is selected from multiple sclerosis and X-linked adrenoleukodystrophy.

Any combination of the groups described above for the various variables is contemplated herein. Throughout the specification, groups and substituents thereof are chosen by one skilled in the field to provide stable moieties and compounds.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Spingosine-1-phospate is a pleiotropic signaling factor that mediates diverse biological responses through interactions with members of the endothelial cell differentiation gene family (EDG receptors) of plasma membrane-localized G protein-coupled receptors. Five members of this family have been identified across a variety of cell types, S1P1 (EDG-1), S1P2 (EDG-5), S1P3 (EDG-3), S1P4 (EDG-6) and S1P5 (EDG-8). The receptor subtypes S1P1, S1P2 and S1P3 are expressed widely in both peripheral tissues and in the central nervous system (CNS) and play key roles in cardiovascular regulation, lymphocyte trafficking and neurogenesis. The S1P4 receptor is expressed mainly in the immune and hematopoietic systems and mediates immune cell proliferation and cytokine production typically in the presence of high concentrations of S1P. The S1P5 receptor expression is notably more restricted and predominantly expressed in oligodendrocytes in the CNS, where it is believed to play a key role in myelin biology, and on natural killer cells and other lymphocytes in the spleen where it is thought to mediate cellular trafficking. S1P5 is also thought to be important in regulating blood-brain barrier permeability and integrity. Oligodendrocyte precursor cells (OPCs), pre-oligodendrocytes, and fully differentiated mature oligodendrocyte cells all express S1P5 receptors at high levels. Activation of the S1P5 receptor can promote the survival of OPCs and drives pre-oligodendrocytes to mature into myelinating Oligodendrocytes. Indeed, preclinical models in mice and xenopus tadpoles have shown evidence of S1P5-mediated remyelination. Additionally, administration of selective S1P5 agonists have been reported to normalize proteinopathies and improve clinical scores in several animal models of neurodegenerative disease. These data support the use of S1P5 agonists in the treatment of a variety of neurological disorders including multiple sclerosis (MS), Alzheimer's disease (AD), Parkinson's disease (PD), Huntington's disease (HD), Neiman Pick Type-C and others.

Fatty acid amide hydrolase (FAAH) is an integral membrane serine hydrolase that degrades the fatty acid amide family of signaling lipids and can hydrolyze select amide prodrugs. FAAH is highly conserved between species and is expressed in many tissues, including the central nervous system (CNS), to varying degrees. Select carboxylic acids can be converted to more permeable amide prodrugs which are then capable of passing through the blood brain barrier where they can be converted to active molecules through the action of FAAH upon the prodrug. This results in the delivery of higher amounts of the carboxylic acid to the CNS as compared to dosing the parent alone. However, peripherally expressed FAAH simultaneously hydrolyzes the prodrug resulting in a considerable amount of non-productive prodrug conversion. Co-administration of a peripherally restricted FAAH inhibitor with a CNS permeable FAAH convertible prodrug increases the selectivity of prodrug delivery to the CNS. It also results in lower exposures of the parent molecule in plasma and peripheral tissue than what is observed when dosing the prodrug alone.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, 4 carbon atoms, 5 carbon atoms, 6 carbon atoms, etc., up to and including 10 carbon atoms, although the present definition also covers the occurrence of the term “alkyl” where no numerical range is designated. In some embodiments, an alkyl is a C₁-C₆alkyl. In one aspect, the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl radical. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. In certain embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises two carbon atoms (e.g., C₂ alkylene). In other embodiments, an alkylene comprises two to four carbon atoms (e.g., C₂-C₄ alkylene). Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Deuteroalkyl” refers to an alkyl group where 1 or more hydrogen atoms of an alkyl are replaced with deuterium.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH—CH₂, —C(CH₃)—CH₂, —CH═CHCH₃, —C(CH₃)—CHCH₃, and —CH₂CH—CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

The term “aromatic” refers to a planar ring having a delocalized x-electron system containing 4n+2 π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon or nitrogen atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycle includes cycloalkyl and aryl.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicyclo[1.1.1]pentyl. In some embodiments, a cycloalkyl is a C₃-C₆cycloalkyl. In some embodiments, a cycloalkyl is a monocyclic cycloalkyl. Monocyclic cycloalkyls include, but are not limited to, cyclopropyl, cyclobutyl, cyclopentyl, cyclohexyl, cycloheptyl, and cyclooctyl. Polycyclic cycloalkyls include, for example, adamantyl, norbornyl (i.e., bicyclo[2.2.2]octyl and bicyclo[2.2.1]heptanyl), norbornenyl, decalinyl, 7,7-dimethyl-bicyclo[2.2.1]heptanyl, and the like.

The term “halo” or, alternatively, “halogen” or “halide” means fluoro, chloro, bromo or iodo. In some embodiments, halo is fluoro, chloro, or bromo.

The term “haloalkyl” refers to an alkyl in which one or more hydrogen atoms are replaced by a halogen atom. In one aspect, a fluoroalkyl is a C₁-C₆fluoroalkyl.

The term “fluoroalkyl” refers to an alkyl in which one or more hydrogen atoms are replaced by a fluorine atom. In one aspect, a fluoroalkyl is a C₁-C₆fluoroalkyl. In some embodiments, a fluoroalkyl is selected from trifluoromethyl, difluoromethyl, fluoromethyl, 2,2,2-trifluoroethyl, 1-fluoromethyl-2-fluoroethyl, and the like.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “heteroalkylene” refers to a divalent heteroalkyl radical.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 2-azabicyclo[2.2.2]octanyl, 3-azabicyclo[3.2.1]octanyl, 5-azabicyclo[2.1.1]hexanyl, 6-azabicyclo[3.1.1]heptanyl, 7-azabicyclo[2.2.1]heptanyl, 8-azabicyclo[3.2.1]octanyl, 3H-indolyl, indolin-2-onyl, isoindolin-1-onyl, isoindoline-1,3-dionyl, 3,4-dihydroisoquinolin-1 (2H)-onyl, 3,4-dihydroquinolin-2 (1H)-onyl, isoindoline-1,3-dithionyl, benzo[d]oxazol-2 (3H)-onyl, 1H-benzo[d]imidazol-2 (3H)-onyl, benzo[d]thiazol-2 (3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl(N-attached) or pyrrol-3-yl(C-attached). Further, a group derived from imidazole includes imidazol-1-yl or imidazol-3-yl(both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-N atoms in the ring. In some embodiments, a heteroaryl contains 0-4 N atoms, 0-1 O atoms, and 0-1 S atoms in the ring. In some embodiments, a heteroaryl contains 1-N atoms, 0-1 O atoms, and 0-1 S atoms in the ring. In some embodiments, heteroaryl is a C₁-C₆heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” or “heteroalicyclic” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “oxo” refers to the ═O radical.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, alkenyl, alkynyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂ (C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O) C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —CH₃, —CH₂CH₃, —CF³, —OCH₃, and —OCF₃.

In some embodiments, substituted groups are substituted with one or two of the preceding groups. In some embodiments, an optional substituent on an aliphatic carbon atom (acyclic or cyclic) includes oxo (═O).

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein, or a pharmaceutically acceptable salt thereof, and a co-agent, are both administered to a patient simultaneously in the form of a single entity or dosage. The term “non-fixed combination” means that the active ingredients, e.g. a compound described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Compounds

The amide compounds Formula (I), (Ia), or (Iaa), described herein, or a pharmaceutically acceptable salt or solvate thereof, are prodrugs of S1P5 receptor agonists.

In some embodiments, described herein is a compound of Formula (I), or a pharmaceutically acceptable salt or solvate thereof:

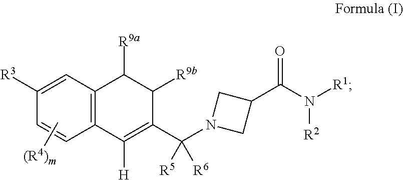

wherein,
R¹ is selected from hydrogen, —OR⁷, —NR⁷R⁸, C₁-C₆alkyl, C₂-C₆alkenyl, C₂-C₆alkynyl, C₃-C₆cycloalkyl, C₂-C₆heterocycloalkyl, C₁₋₉heteroaryl, phenyl, and —C₁-C₆alkyl-phenyl, wherein C₁-C₆alkyl, C₂-C₆alkenyl, C₂-C₆alkynyl, C₃-C₆cycloalkyl, C₂-C₉heterocycloalkyl, C₁₋₉heteroaryl, phenyl, and —C₁-C₆alkyl-phenyl are optionally substituted with one or more groups selected from halogen, C₁-C₆alkyl, C₁₋₆ haloalkyl, C₃-C₆cycloalkyl, —CN, —OR¹⁰, —N(R¹⁰)(R¹¹), and —S(O)₂R¹³;
R² is selected from hydrogen, —OR⁷, —NR⁷R⁸, C₁-C₆alkyl, C₂-C₆alkenyl, C₂-C₆alkynyl, C₃-C₆cycloalkyl, C₂-C₆heterocycloalkyl, C₁₋₉heteroaryl, phenyl, and —C₁-C₆alkyl-phenyl, wherein C₁-C₆alkyl, C₂-C₆alkenyl, C₂-C₆alkynyl, C₃-C₆cycloalkyl, C₂-C₉heterocycloalkyl, C₁₋₉heteroaryl, phenyl, and —C₁-C₆alkyl-phenyl are optionally substituted with one or more groups selected from halogen, C₁-C₆alkyl, C₁₋₆ haloalkyl, C₃-C₆cycloalkyl, —CN, —OR¹⁰, —N(R¹⁰)(R¹¹), and —S(O)₂R¹³; or R¹ and R² are combined to form a C₂-C₉heterocycloalkyl optionally substituted with one or more groups selected from halogen, C₁₋₆alkyl, C₁₋₆haloalkyl, —OR¹⁰, —N(R¹⁰)(R¹¹), and —S(O)₂R¹³;
R³ is selected from halogen, —CN, C₁₋₆alkyl, C₁₋₆haloalkyl, C₂₋₆alkenyl, C₂₋₆alkynyl, —OR¹⁰, —SR¹⁰, —N(R¹⁰)(R¹¹), —C(O)R¹³, —S(O)₂R¹³, and —S(O)₂N(R¹⁰)(R¹¹)—, wherein C₁₋₆ alkyl, C₂₋₆alkenyl, and C₂₋₆alkynyl, are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R⁴ is independently selected from halogen, —CN, C₁₋₆alkyl, C₁₋₆haloalkyl, C₂₋₆ alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²) C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰; R⁵ and R⁶ are independently selected from hydrogen and C₁-C₆alkyl;
each R⁷ and each R⁸ are independently selected from hydrogen and C₁-C₆alkyl; R^{9a} and R^{9b} are independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), wherein C₁₋₆alkyl, C₂₋₆alkenyl, C₂₋₆ alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, —C₁₋₆alkyl-C₃₋₆cycloalkyl, C₂₋₉heterocycloalkyl, —C₁₋₆alkyl-C₂₋₉heterocycloalkyl, C₆₋₁₀aryl, —C₁₋₆alkyl-C₆₋₁₀aryl, C₁₋₉heteroaryl, and —C₁₋₆ alkyl-C₁₋₉heteroaryl, wherein C₁₋₆alkyl, C₂₋₆alkenyl, C₂₋₆alkynyl, C₃₋₆cycloalkyl, —C₁₋₆ alkyl-C₃₋₆cycloalkyl, C₂₋₉heterocycloalkyl, —C₁₋₆alkyl-C₂₋₉heterocycloalkyl, C₆₋₁₀aryl, —C₁₋₆alkyl-C₆₋₁₀aryl, C₁₋₉heteroaryl, and —C₁₋₆alkyl-C₁₋₉heteroaryl are optionally substituted with one, two, or three groups selected from halogen, —CN, hydroxy, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉ heterocycloalkyl, C₆₋₁₀aryl, and C₁₋₉heteroaryl, wherein C₁₋₆alkyl, C₂₋₆alkenyl, C₂₋₆ alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and m is 0, 1, 2, or 3.

For any and all of the embodiments, substituents are selected from among a subset of the listed alternatives. For example, in some embodiments is a compound of Formula (I), or a pharmaceutically acceptable salt or solvate thereof, wherein R⁵ and R⁶ are hydrogen. In some embodiments is a compound of Formula (I), or a pharmaceutically acceptable salt or solvate thereof, wherein R⁵ is C₁₋₆alkyl and R⁶ are hydrogen. In some embodiments is a compound of Formula (I), or a pharmaceutically acceptable salt or solvate thereof, wherein R⁵ and R⁶ are C₁₋₆ alkyl.

In some embodiments is a compound of Formula (I), or a pharmaceutically acceptable salt or solvate thereof, wherein R^{9a} and R^{9b} are independently selected from hydrogen and C₁₋₆ alkyl. In some embodiments is a compound of Formula (I), or a pharmaceutically acceptable salt or solvate thereof, wherein R^{9a} is hydrogen and R^{9b} is C₁₋₆alkyl. In some embodiments is a compound of Formula (I), or a pharmaceutically acceptable salt or solvate thereof, wherein R^{9a} is hydrogen and R^{9b} is —CH₃. In some embodiments is a compound of Formula (I), or a pharmaceutically acceptable salt or solvate thereof, wherein R^{9a} and R^{9b} are C₁₋₆alkyl. In some embodiments is a compound of Formula (I), or a pharmaceutically acceptable salt or solvate thereof, wherein R^{9a} and R^{9b} are —CH₃. In some embodiments is a compound of Formula (I), or a pharmaceutically acceptable salt or solvate thereof, wherein R^{9a} and R^{9b} are hydrogen.

In some embodiments is a compound of Formula (I), or a pharmaceutically acceptable salt or solvate thereof, wherein R³ is selected from C₁₋₆alkyl, C₁₋₆haloalkyl, —OR¹⁰, —SR¹⁰, —N(R¹⁰)(R¹¹), and —C(O)R¹³, wherein C₁₋₆alkyl is optionally substituted with one, two, or three groups selected from halogen, C₁₋₆haloalkyl, —OR¹⁰, and —N(R¹⁰)(R¹¹). In some embodiments is a compound of Formula (I), or a pharmaceutically acceptable salt or solvate thereof, wherein R³ is selected from C₁₋₆alkyl, C₁₋₆haloalkyl, —OR¹⁰, —SR¹⁰, and —N(R¹⁰)(R¹¹), wherein C₁₋₆alkyl is optionally substituted with one group selected from —OR¹⁰ and —N(R¹⁰)(R¹¹). In some embodiments is a compound of Formula (I), or a pharmaceutically acceptable salt or solvate thereof, wherein R³ is C₁₋₆alkyl optionally substituted with one group selected from —OR¹⁰ and —N(R¹⁰)(R¹¹). In some embodiments is a compound of Formula (I), or a pharmaceutically acceptable salt or solvate thereof, wherein R³ is selected from —OR¹⁰, —SR¹⁰, and —N(R¹⁰)(R¹¹), wherein R¹⁰ and R¹¹ are independently selected from hydrogen, C₁₋₆alkyl, and C₁₋₆haloalkyl. In some embodiments is a compound of Formula (I), or a pharmaceutically acceptable salt or solvate thereof, wherein R³ is —OR¹⁰, wherein R¹⁰ is selected from hydrogen, C₁₋₆alkyl, and C₁₋₆ haloalkyl. In some embodiments is a compound of Formula (I), or a pharmaceutically acceptable salt or solvate thereof, wherein R³ is —SR¹⁰, wherein R¹⁰ is selected from hydrogen, C₁₋₆alkyl, and C₁₋₆haloalkyl. In some embodiments is a compound of Formula (I), or a pharmaceutically acceptable salt or solvate thereof, wherein R³ is —N(R¹⁰)(R¹¹), wherein R¹⁰ and R¹¹ are independently selected from hydrogen, C₁₋₆alkyl, and C₁₋₆haloalkyl. In some embodiments is a compound of Formula (I), or a pharmaceutically acceptable salt or solvate thereof, wherein R³ is selected from —O(CH₂)₃CF³, —O(CH₂)₄CF³, —O(CH₂)₅CF³, —S(CH₂)₃CF³, —S(CH₂)₄CF³, —S(CH₂)₅CF³, —N(H)(CH₂)₃CF³, —N(H)(CH₂)₄CF³, and —N(H)(CH₂)₅CF³. In some embodiments is a compound of Formula (I), or a pharmaceutically acceptable salt or solvate thereof, wherein R³ is —O(CH₂)₃CF³. In some embodiments is a compound of Formula (I), or a pharmaceutically acceptable salt or solvate thereof, wherein R³ is —O(CH₂)₄CF³. In some embodiments is a compound of Formula (I), or a pharmaceutically acceptable salt or solvate thereof, wherein R³ is —O(CH₂)₅CF³. In some embodiments is a compound of Formula (I), or a pharmaceutically acceptable salt or solvate thereof, wherein R³ is —S(CH₂)₃CF³. In some embodiments is a compound of Formula (I), or a pharmaceutically acceptable salt or solvate thereof, wherein R³ is —S(CH₂)₄CF³. In some embodiments is a compound of Formula (I), or a pharmaceutically acceptable salt or solvate thereof, wherein R³ is —S(CH₂)₅CF³. In some embodiments is a compound of Formula (I), or a pharmaceutically acceptable salt or solvate thereof, wherein R³ is —N(H)(CH₂)₃CF³. In some embodiments is a compound of Formula (I), or a pharmaceutically acceptable salt or solvate thereof, wherein R³ is —N(H)(CH₂)₄CF³. In some embodiments is a compound of Formula (I), or a pharmaceutically acceptable salt or solvate thereof, wherein R³ is —N(H)(CH₂)₅CF³.

In some embodiments is a compound of Formula (I), or a pharmaceutically acceptable salt or solvate thereof, wherein m is 0.

In some embodiments is a compound of Formula (I), or a pharmaceutically acceptable salt or solvate thereof, wherein m is 1. In some embodiments is a compound of Formula (I), or a pharmaceutically acceptable salt or solvate thereof, wherein m is 2. In some embodiments is a compound of Formula (I), or a pharmaceutically acceptable salt or solvate thereof, wherein m is 3. In some embodiments is a compound of Formula (I), or a pharmaceutically acceptable salt or solvate thereof, wherein each R⁴ is independently selected from halogen, —CN, C₁₋₆alkyl, —OR¹⁰, —SR¹⁰, —N(R¹⁰)(R¹¹), —C(O)OR¹⁰, —C(O)R¹³, —C(O)N(R¹⁰)(R¹¹), —S(O)₂R¹³, and —S(O)₂N(R¹⁰)(R¹¹)—, wherein C₁₋₆alkyl is optionally substituted with one, two, or three groups selected from halogen, C₁₋₆haloalkyl, —OR¹⁰, —N(R¹⁰)(R¹¹), and —C(O)OR¹⁰. In some embodiments is a compound of Formula (I), or a pharmaceutically acceptable salt or solvate thereof, wherein each R⁴ is independently selected from halogen, —CN, C₁₋₆alkyl, —OR¹⁰, and —N(R¹⁰)(R¹¹), wherein C₁₋₆alkyl is optionally substituted with one, two, or three groups selected from halogen, C₁₋₆haloalkyl, —OR¹⁰, —N(R¹⁰)(R¹¹), and —C(O)OR¹⁰. In some embodiments is a compound of Formula (I), or a pharmaceutically acceptable salt or solvate thereof, wherein each R⁴ is independently selected from halogen, C₁₋₆alkyl, —OR¹⁰, wherein C₁₋₆alkyl is optionally substituted with one, two, or three groups selected from halogen, C₁₋₆haloalkyl, and —OR¹⁰.

In some embodiments is a compound of Formula (I), or a pharmaceutically acceptable salt or solvate thereof, wherein R¹ is selected from hydrogen and C₁-C₆alkyl. In some embodiments is a compound of Formula (I), or a pharmaceutically acceptable salt or solvate thereof, wherein R¹ is hydrogen. In some embodiments is a compound of Formula (I), or a pharmaceutically acceptable salt or solvate thereof, wherein R¹ is C₁-C₆alkyl. In some embodiments is a compound of Formula (I), or a pharmaceutically acceptable salt or solvate thereof, wherein R¹ is —CH₃.

In some embodiments is a compound of Formula (I), or a pharmaceutically acceptable salt or solvate thereof, wherein R² is selected from hydrogen, C₁-C₆alkyl, C₂-C₆alkenyl, C₂-C₆alkynyl, C₃-C₆cycloalkyl, C₂-C₆heterocycloalkyl, C₁₋₉heteroaryl, phenyl, and —C₁-C₆alkyl-phenyl, wherein C₁-C₆alkyl, C₂-C₆alkenyl, C₂-C₆alkynyl, C₃-C₆cycloalkyl, C₂-C₉heterocycloalkyl, C₁₋₉heteroaryl, phenyl, and —C₁-C₆alkyl-phenyl are optionally substituted with one or more groups selected from halogen, C₁-C₆alkyl, C₁₋₆haloalkyl, C₃-C₆cycloalkyl, —CN, —OR¹⁰, —N(R¹⁰)(R¹¹), and —S(O)₂R¹³. In some embodiments is a compound of Formula (I), or a pharmaceutically acceptable salt or solvate thereof, wherein R² is selected from hydrogen, C₁-C₆alkyl, C₃-C₆cycloalkyl, C₂-C₆heterocycloalkyl, C₁₋₉heteroaryl, phenyl, and —C₁-C₆alkyl-phenyl, wherein C₁-C₆alkyl, C₃-C₆cycloalkyl, C₂-C₉heterocycloalkyl, C₁₋₉heteroaryl, phenyl, and —C₁-C₆alkyl-phenyl are optionally substituted with one or more groups selected from halogen, C₁-C₆alkyl, C₁₋₆haloalkyl, C₃-C₆cycloalkyl, —CN, —OR¹⁰, —N(R¹⁰)(R¹¹), and —S(O)₂R¹³. In some embodiments is a compound of Formula (I), or a pharmaceutically acceptable salt or solvate thereof, wherein R² is selected from hydrogen, C₁-C₆alkyl, C₃-C₆cycloalkyl, C₂-C₉heterocycloalkyl, C₁₋₉heteroaryl, and phenyl, wherein C₁-C₆alkyl, C₃-C₆cycloalkyl, C₂-C₉heterocycloalkyl, C₁₋₉heteroaryl, and phenyl are optionally substituted with one or more groups selected from halogen, C₁-C₆alkyl, C₁₋₆haloalkyl, C₃-C₆cycloalkyl, —CN, —OR¹⁰, —N(R¹⁰)(R¹¹), and —S(O)₂R¹³. In some embodiments is a compound of Formula (I), or a pharmaceutically acceptable salt or solvate thereof, wherein R² is selected from hydrogen, C₁-C₆alkyl, C₃-C₆cycloalkyl, C₂-C₆heterocycloalkyl, and C₁₋₉heteroaryl, wherein C₁-C₆alkyl, C₃-C₆cycloalkyl, C₂-C₉heterocycloalkyl, and C₁₋₉heteroaryl are optionally substituted with one or more groups selected from halogen, C₁-C₆alkyl, C₁₋₆haloalkyl, C₃-C₆cycloalkyl, —CN, —OR¹⁰, —N(R¹⁰)(R¹¹), and —S(O)₂R¹³.

In some embodiments is a compound of Formula (I), or a pharmaceutically acceptable salt or solvate thereof, wherein R² is C₁-C₆alkyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (I), or a pharmaceutically acceptable salt or solvate thereof, wherein R² is C₁-C₆alkyl optionally substituted with one, two, or three groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (I), or a pharmaceutically acceptable salt or solvate thereof, wherein R² is C₁-C₆alkyl substituted with one or more groups selected from halogen, C₁₋₆haloalkyl, —OR¹⁰, and —N(R¹⁰)(R¹¹). In some embodiments is a compound of Formula (I), or a pharmaceutically acceptable salt or solvate thereof, wherein R² is C₁-C₆alkyl substituted with one, two, or three groups selected from halogen, C₁₋₆haloalkyl, —OR¹⁰, and —N(R¹⁰)(R¹¹). In some embodiments is a compound of Formula (I), or a pharmaceutically acceptable salt or solvate thereof, wherein R² is C₁-C₆alkyl substituted with one, two, or three halogen. In some embodiments is a compound of Formula (I), or a pharmaceutically acceptable salt or solvate thereof, wherein R² is C₁-C₆alkyl substituted with one, two, or three C₁₋₆haloalkyl. In some embodiments is a compound of Formula (I), or a pharmaceutically acceptable salt or solvate thereof, wherein R² is C₁-C₆alkyl substituted with one, two, or three —OR¹⁰. In some embodiments is a compound of Formula (I), or a pharmaceutically acceptable salt or solvate thereof, wherein R² is C₁-C₆alkyl substituted with one, two, or three —OH. In some embodiments is a compound of Formula (I), or a pharmaceutically acceptable salt or solvate thereof, wherein R² is C₁-C₆alkyl substituted with one, two, or three —N(R¹⁰)(R¹¹).

In some embodiments is a compound of Formula (I), or a pharmaceutically acceptable salt or solvate thereof, wherein R² is unsubstituted C₁-C₆alkyl. In some embodiments is a compound of Formula (I), or a pharmaceutically acceptable salt or solvate thereof, wherein R² is —CH₃. In some embodiments is a compound of Formula (I), or a pharmaceutically acceptable salt or solvate thereof, wherein R² is —CH₂CH₃.

In some embodiments is a compound of Formula (I), or a pharmaceutically acceptable salt or solvate thereof, wherein R² is hydrogen.

In some embodiments is a compound of Formula (I), or a pharmaceutically acceptable salt or solvate thereof, wherein R² is C₃-C₆cycloalkyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (I), or a pharmaceutically acceptable salt or solvate thereof, wherein R² is C₃-C₆cycloalkyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (I), or a pharmaceutically acceptable salt or solvate thereof, wherein R² is C₃-C₆cycloalkyl optionally substituted with one, two, or three groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (I), or a pharmaceutically acceptable salt or solvate thereof, wherein R² is C₃-C₆cycloalkyl substituted with one or more groups selected from halogen, C₁₋₆haloalkyl, —OR¹⁰, and —N(R¹⁰)(R¹¹). In some embodiments is a compound of Formula (I), or a pharmaceutically acceptable salt or solvate thereof, wherein R² is C₃-C₆cycloalkyl substituted with one, two, or three groups selected from halogen, C₁₋₆haloalkyl, —OR¹⁰, and —N(R¹⁰)(R¹¹). In some embodiments is a compound of Formula (I), or a pharmaceutically acceptable salt or solvate thereof, wherein R² is C₁-C₆alkyl substituted with one, two, or three halogen. In some embodiments is a compound of Formula (I), or a pharmaceutically acceptable salt or solvate thereof, wherein R² is C₃-C₆cycloalkyl substituted with one, two, or three C₁₋₆haloalkyl. In some embodiments is a compound of Formula (I), or a pharmaceutically acceptable salt or solvate thereof, wherein R² is C₃-C₆cycloalkyl substituted with one, two, or three —OR¹⁰. In some embodiments is a compound of Formula (I), or a pharmaceutically acceptable salt or solvate thereof, wherein R² is C₃-C₆cycloalkyl substituted with one, two, or three —OH. In some embodiments is a compound of Formula (I), or a pharmaceutically acceptable salt or solvate thereof, wherein R² is C₃-C₆cycloalkyl substituted with one, two, or three —N(R¹⁰)(R¹¹).

In some embodiments is a compound of Formula (I), or a pharmaceutically acceptable salt or solvate thereof, wherein R² is unsubstituted C₃-C₆cycloalkyl. In some embodiments is a compound of Formula (I), or a pharmaceutically acceptable salt or solvate thereof, wherein R² is unsubstituted cyclopropyl. In some embodiments is a compound of Formula (I), or a pharmaceutically acceptable salt or solvate thereof, wherein R² is unsubstituted cyclobutyl. In some embodiments is a compound of Formula (I), or a pharmaceutically acceptable salt or solvate thereof, wherein R² is unsubstituted cyclopentyl. In some embodiments is a compound of Formula (I), or a pharmaceutically acceptable salt or solvate thereof, wherein R² is unsubstituted cyclohexyl.

In some embodiments is a compound of Formula (I), or a pharmaceutically acceptable salt or solvate thereof, wherein R² is C₁₋₉heteroaryl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (I), or a pharmaceutically acceptable salt or solvate thereof, wherein R² is C₁₋₉heteroaryl optionally substituted with one or more groups selected from halogen, C₁-C₆alkyl, C₁₋₆haloalkyl, —CN, —OR¹⁰, —N(R¹⁰)(R¹¹), and —S(O)₂R¹³, wherein C₁₋₉heteroaryl is selected from pyridyl, pyridazinyl, pyrimidinyl, and pyrazinyl. In some embodiments is a compound of Formula (I), or a pharmaceutically acceptable salt or solvate thereof, wherein R² is pyridyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (I), or a pharmaceutically acceptable salt or solvate thereof, wherein R² is pyridazinyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (I), or a pharmaceutically acceptable salt or solvate thereof, wherein R² is pyrimidinyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (I), or a pharmaceutically acceptable salt or solvate thereof, wherein R² is pyrazinyl optionally substituted with one or more groups selected from halogen, C₁-C₆alkyl, C₁₋₆haloalkyl, —CN, —OR¹⁰, —N(R¹⁰)(R¹¹), and —S(O)₂R¹³.

In some embodiments is a compound of Formula (I), or a pharmaceutically acceptable salt or solvate thereof, wherein R² is selected from unsubstituted pyridyl, unsubstituted pyridazinyl, unsubstituted pyrimidinyl, and unsubstituted pyrazinyl. In some embodiments is a compound of Formula (I), or a pharmaceutically acceptable salt or solvate thereof, wherein R² is unsubstituted pyridyl. In some embodiments is a compound of Formula (I), or a pharmaceutically acceptable salt or solvate thereof, wherein R² is unsubstituted pyridazinyl. In some embodiments is a compound of Formula (I), or a pharmaceutically acceptable salt or solvate thereof, wherein R² is unsubstituted pyrimidinyl. In some embodiments is a compound of Formula (I), or a pharmaceutically acceptable salt or solvate thereof, wherein R² is unsubstituted pyrazinyl.

In some embodiments is a compound of Formula (I), or a pharmaceutically acceptable salt or solvate thereof, wherein R¹ and R² are combined to form a C₂-C₉heterocycloalkyl optionally substituted with one or more groups selected from halogen, C₁₋₆alkyl, C₁₋₆haloalkyl, —OR¹⁰, —N(R¹⁰)(R¹¹), and —S(O)₂R¹³. In some embodiments is a compound of Formula (I), or a pharmaceutically acceptable salt or solvate thereof, wherein R¹ and R² are combined to form a C₂-C₉heterocycloalkyl optionally substituted with one, two, or three groups selected from halogen, C₁₋₆alkyl, C₁₋₆haloalkyl, —OR¹⁰, —N(R¹⁰)(R¹¹), and —S(O)₂R¹³. In some embodiments is a compound of Formula (I), or a pharmaceutically acceptable salt or solvate thereof, wherein R¹ and R² are combined to form an unsubstituted C₂-C₆heterocycloalkyl.

In some embodiments, described herein is a compound of Formula (Ia), or a pharmaceutically acceptable salt or solvate thereof:

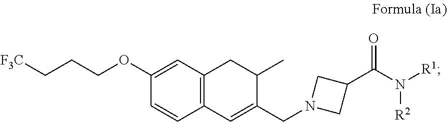

wherein,
R¹ is selected from hydrogen, —OR⁷, —NR⁷R⁸, C₁-C₆alkyl, C₂-C₆alkenyl, C₂-C₆alkynyl, C₃-C₆cycloalkyl, C₂-C₆heterocycloalkyl, C₁₋₉heteroaryl, phenyl, and —C₁-C₆alkyl-phenyl, wherein C₁-C₆alkyl, C₂-C₆alkenyl, C₂-C₆alkynyl, C₃-C₆cycloalkyl, C₂-C₉heterocycloalkyl, C₁₋₉heteroaryl, phenyl, and —C₁-C₆alkyl-phenyl are optionally substituted with one or more groups selected from halogen, C₁-C₆alkyl, C₁₋₆ haloalkyl, C₃-C₆cycloalkyl, —CN, —OR¹⁰, —N(R¹⁰)(R¹¹), and —S(O)₂R¹³;
R² is selected from hydrogen, —OR⁷, —NR⁷R⁸, C₁-C₆alkyl, C₂-C₆alkenyl, C₂-C₆alkynyl, C₃-C₆cycloalkyl, C₂-C₆heterocycloalkyl, C₁₋₉heteroaryl, phenyl, and —C₁-C₆alkyl-phenyl, wherein C₁-C₆alkyl, C₂-C₆alkenyl, C₂-C₆alkynyl, C₃-C₆cycloalkyl, C₂-C₉heterocycloalkyl, C₁₋₉heteroaryl, phenyl, and —C₁-C₆alkyl-phenyl are optionally substituted with one or more groups selected from halogen, C₁-C₆alkyl, C₁₋₆ haloalkyl, C₃-C₆cycloalkyl, —CN, —OR¹⁰, —N(R¹⁰)(R¹¹), and —S(O)₂R¹³; or R¹ and R² are combined to form a C₂-C₉heterocycloalkyl optionally substituted with one or more groups selected from halogen, C₁₋₆alkyl, C₁₋₆haloalkyl, —OR¹⁰, —N(R¹⁰)(R¹¹), and —S(O)₂R¹³;
each R⁷ and each R⁸ are independently selected from hydrogen and C₁-C₆alkyl;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, —C₁₋₆alkyl-C₃₋₆cycloalkyl, C₂₋₉heterocycloalkyl, —C₁₋₆ alkyl-C₂₋₉heterocycloalkyl, C₆₋₁₀aryl, —C₁₋₆alkyl-C₆₋₁₀aryl, C₁₋₉heteroaryl, and —C₁₋₆ alkyl-C₁₋₉heteroaryl, wherein C₁₋₆alkyl, C₂₋₆alkenyl, C₂₋₆alkynyl, C₃₋₆cycloalkyl, —C₁₋₆ alkyl-C₃₋₆cycloalkyl, C₂₋₉heterocycloalkyl, —C₁₋₆alkyl-C₂₋₉heterocycloalkyl, C₆₋₁₀aryl, —C₁₋₆alkyl-C₆₋₁₀aryl, C₁₋₉heteroaryl, and —C₁₋₆alkyl-C₁₋₉heteroaryl are optionally substituted with one, two, or three groups selected from halogen, —CN, hydroxy, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉ heterocycloalkyl, C₆₋₁₀aryl, and C₁₋₉heteroaryl, wherein C₁₋₆alkyl, C₂₋₆alkenyl, C₂₋₆ alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In some embodiments is a compound of Formula (Ia), or a pharmaceutically acceptable salt or solvate thereof, wherein R¹ is selected from hydrogen and C₁-C₆alkyl. In some embodiments is a compound of Formula (Ia), or a pharmaceutically acceptable salt or solvate thereof, wherein R¹ is hydrogen. In some embodiments is a compound of Formula (Ia), or a pharmaceutically acceptable salt or solvate thereof, wherein R¹ is C₁-C₆alkyl. In some embodiments is a compound of Formula (Ia), or a pharmaceutically acceptable salt or solvate thereof, wherein R¹ is —CH₃.

In some embodiments is a compound of Formula (Ia), or a pharmaceutically acceptable salt or solvate thereof, wherein R² is selected from hydrogen, C₁-C₆alkyl, C₂-C₆alkenyl, C₂-C₆alkynyl, C₃-C₆cycloalkyl, C₂-C₉heterocycloalkyl, C₁₋₉heteroaryl, phenyl, and —C₁-C₆alkyl-phenyl, wherein C₁-C₆alkyl, C₂-C₆alkenyl, C₂-C₆alkynyl, C₃-C₆cycloalkyl, C₂-C₉heterocycloalkyl, C₁₋₉heteroaryl, phenyl, and —C₁-C₆alkyl-phenyl are optionally substituted with one or more groups selected from halogen, C₁-C₆alkyl, C₁₋₆haloalkyl, C₃-C₆cycloalkyl, —CN, —OR¹⁰, —N(R¹⁰)(R¹¹), and —S(O)₂R¹³. In some embodiments is a compound of Formula (Ia), or a pharmaceutically acceptable salt or solvate thereof, wherein R² is selected from hydrogen, C₁-C₆alkyl, C₃-C₆cycloalkyl, C₂-C₉heterocycloalkyl, C₁₋₉heteroaryl, phenyl, and —C₁-C₆alkyl-phenyl, wherein C₁-C₆alkyl, C₃-C₆cycloalkyl, C₂-C₉heterocycloalkyl, C₁₋₉heteroaryl, phenyl, and —C₁-C₆alkyl-phenyl are optionally substituted with one or more groups selected from halogen, C₁-C₆alkyl, C₁₋₆haloalkyl, C₃-C₆cycloalkyl, —CN, —OR¹⁰, —N(R¹⁰)(R¹¹), and —S(O)₂R¹³. In some embodiments is a compound of Formula (Ia), or a pharmaceutically acceptable salt or solvate thereof, wherein R² is selected from hydrogen, C₁-C₆alkyl, C₃-C₆cycloalkyl, C₂-C₉heterocycloalkyl, C₁₋₉heteroaryl, and phenyl, wherein C₁-C₆alkyl, C₃-C₆cycloalkyl, C₂-C₉heterocycloalkyl, C₁₋₉heteroaryl, and phenyl are optionally substituted with one or more groups selected from halogen, C₁-C₆alkyl, C₁₋₆haloalkyl, C₃-C₆cycloalkyl, —CN, —OR¹⁰, —N(R¹⁰)(R¹¹), and —S(O)₂R¹³. In some embodiments is a compound of Formula (Ia), or a pharmaceutically acceptable salt or solvate thereof, wherein R² is selected from hydrogen, C₁-C₆alkyl, C₃-C₆cycloalkyl, C₂-C₆heterocycloalkyl, and C₁₋₉heteroaryl, wherein C₁-C₆alkyl, C₃-C₆cycloalkyl, C₂-C₆heterocycloalkyl, and C₁₋₉heteroaryl are optionally substituted with one or more groups selected from halogen, C₁-C₆alkyl, C₁₋₆haloalkyl, C₃-C₆cycloalkyl, —CN, —OR¹⁰, —N(R¹⁰)(R¹¹), and —S(O)₂R¹³.

In some embodiments is a compound of Formula (Ia), or a pharmaceutically acceptable salt or solvate thereof, wherein R² is C₁-C₆alkyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Ia), or a pharmaceutically acceptable salt or solvate thereof, wherein R² is C₁-C₆alkyl optionally substituted with one, two, or three groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Ia), or a pharmaceutically acceptable salt or solvate thereof, wherein R² is C₁-C₆alkyl substituted with one or more groups selected from halogen, C₁₋₆haloalkyl, —OR¹⁰, and —N(R¹⁰)(R¹¹). In some embodiments is a compound of Formula (Ia), or a pharmaceutically acceptable salt or solvate thereof, wherein R² is C₁-C₆alkyl substituted with one, two, or three groups selected from halogen, C₁₋₆haloalkyl, —OR¹⁰, and —N(R¹⁰)(R¹¹). In some embodiments is a compound of Formula (Ia), or a pharmaceutically acceptable salt or solvate thereof, wherein R² is C₁-C₆alkyl substituted with one, two, or three halogen. In some embodiments is a compound of Formula (Ia), or a pharmaceutically acceptable salt or solvate thereof, wherein R² is C₁-C₆alkyl substituted with one, two, or three C₁₋₆haloalkyl. In some embodiments is a compound of Formula (Ia), or a pharmaceutically acceptable salt or solvate thereof, wherein R² is C₁-C₆alkyl substituted with one, two, or three —OR¹⁰. In some embodiments is a compound of Formula (Ia), or a pharmaceutically acceptable salt or solvate thereof, wherein R² is C₁-C₆alkyl substituted with one, two, or three —OH. In some embodiments is a compound of Formula (Ia), or a pharmaceutically acceptable salt or solvate thereof, wherein R² is C₁-C₆alkyl substituted with one, two, or three —N(R¹⁰)(R¹¹).

In some embodiments is a compound of Formula (Ia), or a pharmaceutically acceptable salt or solvate thereof, wherein R² is unsubstituted C₁-C₆alkyl. In some embodiments is a compound of Formula (Ia), or a pharmaceutically acceptable salt or solvate thereof, wherein R² is —CH₃. In some embodiments is a compound of Formula (Ia), or a pharmaceutically acceptable salt or solvate thereof, wherein R² is —CH₂CH₃.

In some embodiments is a compound of Formula (Ia), or a pharmaceutically acceptable salt or solvate thereof, wherein R² is hydrogen.

In some embodiments is a compound of Formula (Ia), or a pharmaceutically acceptable salt or solvate thereof, wherein R² is C₃-C₆cycloalkyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Ia), or a pharmaceutically acceptable salt or solvate thereof, wherein R² is C₃-C₆cycloalkyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Ia), or a pharmaceutically acceptable salt or solvate thereof, wherein R² is C₃-C₆cycloalkyl optionally substituted with one, two, or three groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Ia), or a pharmaceutically acceptable salt or solvate thereof, wherein R² is C₃-C₆cycloalkyl substituted with one or more groups selected from halogen, C₁₋₆haloalkyl, —OR¹⁰, and —N(R¹⁰)(R¹¹). In some embodiments is a compound of Formula (Ia), or a pharmaceutically acceptable salt or solvate thereof, wherein R² is C₃-C₆cycloalkyl substituted with one, two, or three groups selected from halogen, C₁₋₆ haloalkyl, —OR¹⁰, and —N(R¹⁰)(R¹¹). In some embodiments is a compound of Formula (Ia), or a pharmaceutically acceptable salt or solvate thereof, wherein R² is C₁-C₆alkyl substituted with one, two, or three halogen. In some embodiments is a compound of Formula (Ia), or a pharmaceutically acceptable salt or solvate thereof, wherein R² is C₃-C₆cycloalkyl substituted with one, two, or three C₁₋₆haloalkyl. In some embodiments is a compound of Formula (Ia), or a pharmaceutically acceptable salt or solvate thereof, wherein R² is C₃-C₆cycloalkyl substituted with one, two, or three —OR¹⁰. In some embodiments is a compound of Formula (Ia), or a pharmaceutically acceptable salt or solvate thereof, wherein R² is C₃-C₆cycloalkyl substituted with one, two, or three —OH. In some embodiments is a compound of Formula (Ia), or a pharmaceutically acceptable salt or solvate thereof, wherein R² is C₃-C₆cycloalkyl substituted with one, two, or three —N(R¹⁰)(R¹¹).

In some embodiments is a compound of Formula (Ia), or a pharmaceutically acceptable salt or solvate thereof, wherein R² is unsubstituted C₃-C₆cycloalkyl. In some embodiments is a compound of Formula (Ia), or a pharmaceutically acceptable salt or solvate thereof, wherein R² is unsubstituted cyclopropyl. In some embodiments is a compound of Formula (Ia), or a pharmaceutically acceptable salt or solvate thereof, wherein R² is unsubstituted cyclobutyl. In some embodiments is a compound of Formula (Ia), or a pharmaceutically acceptable salt or solvate thereof, wherein R² is unsubstituted cyclopentyl. In some embodiments is a compound of Formula (Ia), or a pharmaceutically acceptable salt or solvate thereof, wherein R² is unsubstituted cyclohexyl.

In some embodiments is a compound of Formula (Ia), or a pharmaceutically acceptable salt or solvate thereof, wherein R² is C₁₋₉heteroaryl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Ia), or a pharmaceutically acceptable salt or solvate thereof, wherein R² is C₁₋₉heteroaryl optionally substituted with one or more groups selected from halogen, C₁-C₆alkyl, C₁₋₆haloalkyl, —CN, —OR¹⁰, —N(R¹⁰)(R¹¹), and —S(O)₂R¹³, wherein C₁₋₉heteroaryl is selected from pyridyl, pyridazinyl, pyrimidinyl, and pyrazinyl. In some embodiments is a compound of Formula (Ia), or a pharmaceutically acceptable salt or solvate thereof, wherein R² is pyridyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Ia), or a pharmaceutically acceptable salt or solvate thereof, wherein R² is pyridazinyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Ia), or a pharmaceutically acceptable salt or solvate thereof, wherein R² is pyrimidinyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Ia), or a pharmaceutically acceptable salt or solvate thereof, wherein R² is pyrazinyl optionally substituted with one or more groups selected from halogen, C₁-C₆alkyl, C₁₋₆haloalkyl, —CN, —OR¹⁰, —N(R¹⁰)(R¹¹), and —S(O)₂R¹³.

In some embodiments is a compound of Formula (Ia), or a pharmaceutically acceptable salt or solvate thereof, wherein R² is selected from unsubstituted pyridyl, unsubstituted pyridazinyl, unsubstituted pyrimidinyl, and unsubstituted pyrazinyl. In some embodiments is a compound of Formula (Ia), or a pharmaceutically acceptable salt or solvate thereof, wherein R² is unsubstituted pyridyl. In some embodiments is a compound of Formula (Ia), or a pharmaceutically acceptable salt or solvate thereof, wherein R² is unsubstituted pyridazinyl. In some embodiments is a compound of Formula (Ia), or a pharmaceutically acceptable salt or solvate thereof, wherein R² is unsubstituted pyrimidinyl. In some embodiments is a compound of Formula (Ia), or a pharmaceutically acceptable salt or solvate thereof, wherein R² is unsubstituted pyrazinyl.

In some embodiments is a compound of Formula (Ia), or a pharmaceutically acceptable salt or solvate thereof, wherein R¹ and R² are combined to form a C₂-C₉heterocycloalkyl optionally substituted with one or more groups selected from halogen, C₁₋₆alkyl, C₁₋₆haloalkyl, —OR¹⁰, —N(R¹⁰)(R¹¹), and —S(O)₂R¹³. In some embodiments is a compound of Formula (Ia), or a pharmaceutically acceptable salt or solvate thereof, wherein R¹ and R² are combined to form a C₂-C₉heterocycloalkyl optionally substituted with one, two, or three groups selected from halogen, C₁₋₆alkyl, C₁₋₆haloalkyl, —OR¹⁰, —N(R¹⁰)(R¹¹), and —S(O)₂R¹³. In some embodiments is a compound of Formula (Ia), or a pharmaceutically acceptable salt or solvate thereof, wherein R¹ and R² are combined to form an unsubstituted C₂-C₆heterocycloalkyl.

In some embodiments, described herein is a compound of Formula (Iaa), or a pharmaceutically acceptable salt or solvate thereof:

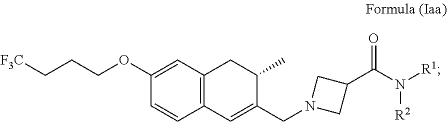

wherein,
R¹ is selected from hydrogen, —OR⁷, —NR⁷R⁸, C₁-C₆alkyl, C₂-C₆alkenyl, C₂-C₆alkynyl, C₃-C₆cycloalkyl, C₂-C₆heterocycloalkyl, C₁₋₉heteroaryl, phenyl, and —C₁-C₆alkyl-phenyl, wherein C₁-C₆alkyl, C₂-C₆alkenyl, C₂-C₆alkynyl, C₃-C₆cycloalkyl, C₂-C₉heterocycloalkyl, C₁₋₉heteroaryl, phenyl, and —C₁-C₆alkyl-phenyl are optionally substituted with one or more groups selected from halogen, C₁-C₆alkyl, C₁₋₆ haloalkyl, C₃-C₆cycloalkyl, —CN, —OR¹⁰, —N(R¹⁰)(R¹¹), and —S(O)₂R¹³;
R² is selected from hydrogen, —OR⁷, —NR⁷R⁸, C₁-C₆alkyl, C₂-C₆alkenyl, C₂-C₆alkynyl, C₃-C₆cycloalkyl, C₂-C₆heterocycloalkyl, C₁₋₉heteroaryl, phenyl, and —C₁-C₆alkyl-phenyl, wherein C₁-C₆alkyl, C₂-C₆alkenyl, C₂-C₆alkynyl, C₃-C₆cycloalkyl, C₂-C₉heterocycloalkyl, C₁₋₉heteroaryl, phenyl, and —C₁-C₆alkyl-phenyl are optionally substituted with one or more groups selected from halogen, C₁-C₆alkyl, C₁₋₆ haloalkyl, C₃-C₆cycloalkyl, —CN, —OR¹⁰, —N(R¹⁰)(R¹¹), and —S(O)₂R¹³; or R¹ and R² are combined to form a C₂-C₉heterocycloalkyl optionally substituted with one or more groups selected from halogen, C₁₋₆alkyl, C₁₋₆haloalkyl, —OR¹⁰, —N(R¹⁰)(R¹¹), and —S(O)₂R¹³,
each R⁷ and each R⁸ are independently selected from hydrogen and C₁-C₆alkyl;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, —C₁₋₆alkyl-C₃₋₆cycloalkyl, C₂₋₉heterocycloalkyl, —C₁₋₆ alkyl-C₂₋₉heterocycloalkyl, C₆₋₁₀aryl, —C₁₋₆alkyl-C₆₋₁₀aryl, C₁₋₉heteroaryl, and —C₁₋₆ alkyl-C₁₋₉heteroaryl, wherein C₁₋₆alkyl, C₂₋₆alkenyl, C₂₋₆alkynyl, C₃₋₆cycloalkyl, —C₁₋₆ alkyl-C₃₋₆cycloalkyl, C₂₋₉heterocycloalkyl, —C₁₋₆alkyl-C₂₋₉heterocycloalkyl, C₆₋₁₀aryl, —C₁₋₆alkyl-C₆₋₁₀aryl, C₁₋₉heteroaryl, and —C₁₋₆alkyl-C₁₋₉heteroaryl are optionally substituted with one, two, or three groups selected from halogen, —CN, hydroxy, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉ heterocycloalkyl, C₆₋₁₀aryl, and C₁₋₉heteroaryl, wherein C₁₋₆alkyl, C₂₋₆alkenyl, C₂₋₆ alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In some embodiments is a compound of Formula (Iaa), or a pharmaceutically acceptable salt or solvate thereof, wherein R¹ is selected from hydrogen and C₁-C₆alkyl. In some embodiments is a compound of Formula (Iaa), or a pharmaceutically acceptable salt or solvate thereof, wherein R¹ is hydrogen. In some embodiments is a compound of Formula (Iaa), or a pharmaceutically acceptable salt or solvate thereof, wherein R¹ is C₁-C₆alkyl. In some embodiments is a compound of Formula (Iaa), or a pharmaceutically acceptable salt or solvate thereof, wherein R¹ is —CH₃.

In some embodiments is a compound of Formula (Iaa), or a pharmaceutically acceptable salt or solvate thereof, wherein R² is selected from hydrogen, C₁-C₆alkyl, C₂-C₆alkenyl, C₂-C₆alkynyl, C₃-C₆cycloalkyl, C₂-C₉heterocycloalkyl, C₁₋₉heteroaryl, phenyl, and —C₁-C₆alkyl-phenyl, wherein C₁-C₆alkyl, C₂-C₆alkenyl, C₂-C₆alkynyl, C₃-C₆cycloalkyl, C₂-C₉heterocycloalkyl, C₁₋₉heteroaryl, phenyl, and —C₁-C₆alkyl-phenyl are optionally substituted with one or more groups selected from halogen, C₁-C₆alkyl, C₁₋₆haloalkyl, C₃-C₆cycloalkyl, —CN, —OR¹⁰, —N(R¹⁰)(R¹¹), and —S(O)₂R¹³. In some embodiments is a compound of Formula (Iaa), or a pharmaceutically acceptable salt or solvate thereof, wherein R² is selected from hydrogen, C₁-C₆alkyl, C₃-C₆cycloalkyl, C₂-C₉heterocycloalkyl, C₁₋₉heteroaryl, phenyl, and —C₁-C₆alkyl-phenyl, wherein C₁-C₆alkyl, C₃-C₆cycloalkyl, C₂-C₆heterocycloalkyl, C₁₋₉heteroaryl, phenyl, and —C₁-C₆alkyl-phenyl are optionally substituted with one or more groups selected from halogen, C₁-C₆alkyl, C₁₋₆haloalkyl, C₃-C₆cycloalkyl, —CN, —OR¹⁰, —N(R¹⁰)(R¹¹), and —S(O)₂R¹³. In some embodiments is a compound of Formula (Iaa), or a pharmaceutically acceptable salt or solvate thereof, wherein R² is selected from hydrogen, C₁-C₆alkyl, C₃-C₆cycloalkyl, C₂-C₉heterocycloalkyl, C₁₋₉heteroaryl, and phenyl, wherein C₁-C₆alkyl, C₃-C₆cycloalkyl, C₂-C₉heterocycloalkyl, C₁₋₉heteroaryl, and phenyl are optionally substituted with one or more groups selected from halogen, C₁-C₆alkyl, C₁₋₆haloalkyl, C₃-C₆cycloalkyl, —CN, —OR¹⁰, —N(R¹⁰)(R¹¹), and —S(O)₂R¹³. In some embodiments is a compound of Formula (Iaa), or a pharmaceutically acceptable salt or solvate thereof, wherein R² is selected from hydrogen, C₁-C₆alkyl, C₃-C₆cycloalkyl, C₂-C₆heterocycloalkyl, and C₁₋₉heteroaryl, wherein C₁-C₆alkyl, C₃-C₆cycloalkyl, C₂-C₉heterocycloalkyl, and C₁₋₉heteroaryl are optionally substituted with one or more groups selected from halogen, C₁-C₆alkyl, C₁₋₆haloalkyl, C₃-C₆cycloalkyl, —CN, —OR¹⁰, —N(R¹⁰)(R¹¹), and —S(O)₂R¹³.

In some embodiments is a compound of Formula (Iaa), or a pharmaceutically acceptable salt or solvate thereof, wherein R² is C₁-C₆alkyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Iaa), or a pharmaceutically acceptable salt or solvate thereof, wherein R² is C₁-C₆alkyl optionally substituted with one, two, or three groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Iaa), or a pharmaceutically acceptable salt or solvate thereof, wherein R² is C₁-C₆alkyl substituted with one or more groups selected from halogen, C₁₋₆haloalkyl, —OR¹⁰, and —N(R¹⁰)(R¹¹). In some embodiments is a compound of Formula (Iaa), or a pharmaceutically acceptable salt or solvate thereof, wherein R² is C₁-C₆alkyl substituted with one, two, or three groups selected from halogen, C₁₋₆haloalkyl, —OR¹⁰, and —N(R¹⁰)(R¹¹). In some embodiments is a compound of Formula (Iaa), or a pharmaceutically acceptable salt or solvate thereof, wherein R² is C₁-C₆alkyl substituted with one, two, or three halogen. In some embodiments is a compound of Formula (Iaa), or a pharmaceutically acceptable salt or solvate thereof, wherein R² is C₁-C₆alkyl substituted with one, two, or three C₁₋₆haloalkyl. In some embodiments is a compound of Formula (Iaa), or a pharmaceutically acceptable salt or solvate thereof, wherein R² is C₁-C₆alkyl substituted with one, two, or three —OR¹⁰. In some embodiments is a compound of Formula (Iaa), or a pharmaceutically acceptable salt or solvate thereof, wherein R² is C₁-C₆alkyl substituted with one, two, or three —OH. In some embodiments is a compound of Formula (Iaa), or a pharmaceutically acceptable salt or solvate thereof, wherein R² is C₁-C₆alkyl substituted with one, two, or three —N(R¹⁰)(R¹¹).

In some embodiments is a compound of Formula (Iaa), or a pharmaceutically acceptable salt or solvate thereof, wherein R² is unsubstituted C₁-C₆alkyl. In some embodiments is a compound of Formula (Iaa), or a pharmaceutically acceptable salt or solvate thereof, wherein R² is —CH₃. In some embodiments is a compound of Formula (Iaa), or a pharmaceutically acceptable salt or solvate thereof, wherein R² is —CH₂CH₃.

In some embodiments is a compound of Formula (Iaa), or a pharmaceutically acceptable salt or solvate thereof, wherein R² is hydrogen.

In some embodiments is a compound of Formula (Iaa), or a pharmaceutically acceptable salt or solvate thereof, wherein R² is C₃-C₆cycloalkyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Iaa), or a pharmaceutically acceptable salt or solvate thereof, wherein R² is C₃-C₆cycloalkyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Iaa), or a pharmaceutically acceptable salt or solvate thereof, wherein R² is C₃-C₆cycloalkyl optionally substituted with one, two, or three groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Iaa), or a pharmaceutically acceptable salt or solvate thereof, wherein R² is C₃-C₆cycloalkyl substituted with one or more groups selected from halogen, C₁₋₆haloalkyl, —OR¹⁰, and —N(R¹⁰)(R¹¹). In some embodiments is a compound of Formula (Iaa), or a pharmaceutically acceptable salt or solvate thereof, wherein R² is C₃-C₆cycloalkyl substituted with one, two, or three groups selected from halogen, C₁₋₆ haloalkyl, —OR¹⁰, and —N(R¹⁰)(R¹¹). In some embodiments is a compound of Formula (Iaa), or a pharmaceutically acceptable salt or solvate thereof, wherein R² is C₁-C₆alkyl substituted with one, two, or three halogen. In some embodiments is a compound of Formula (Iaa), or a pharmaceutically acceptable salt or solvate thereof, wherein R² is C₃-C₆cycloalkyl substituted with one, two, or three C₁₋₆haloalkyl. In some embodiments is a compound of Formula (Iaa), or a pharmaceutically acceptable salt or solvate thereof, wherein R² is C₃-C₆cycloalkyl substituted with one, two, or three —OR¹⁰. In some embodiments is a compound of Formula (Iaa), or a pharmaceutically acceptable salt or solvate thereof, wherein R² is C₃-C₆cycloalkyl substituted with one, two, or three —OH. In some embodiments is a compound of Formula (Iaa), or a pharmaceutically acceptable salt or solvate thereof, wherein R² is C₃-C₆cycloalkyl substituted with one, two, or three —N(R¹⁰)(R¹¹).

In some embodiments is a compound of Formula (Iaa), or a pharmaceutically acceptable salt or solvate thereof, wherein R² is unsubstituted C₃-C₆cycloalkyl. In some embodiments is a compound of Formula (Iaa), or a pharmaceutically acceptable salt or solvate thereof, wherein R² is unsubstituted cyclopropyl. In some embodiments is a compound of Formula (Iaa), or a pharmaceutically acceptable salt or solvate thereof, wherein R² is unsubstituted cyclobutyl. In some embodiments is a compound of Formula (Iaa), or a pharmaceutically acceptable salt or solvate thereof, wherein R² is unsubstituted cyclopentyl. In some embodiments is a compound of Formula (Iaa), or a pharmaceutically acceptable salt or solvate thereof, wherein R² is unsubstituted cyclohexyl.

In some embodiments is a compound of Formula (Iaa), or a pharmaceutically acceptable salt or solvate thereof, wherein R² is C₁₋₉heteroaryl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Iaa), or a pharmaceutically acceptable salt or solvate thereof, wherein R² is C₁₋₉heteroaryl optionally substituted with one or more groups selected from halogen, C₁-C₆alkyl, C₁₋₆haloalkyl, —CN, —OR¹⁰, —N(R¹⁰)(R¹¹), and —S(O)₂R¹³, wherein C₁₋₉heteroaryl is selected from pyridyl, pyridazinyl, pyrimidinyl, and pyrazinyl. In some embodiments is a compound of Formula (Iaa), or a pharmaceutically acceptable salt or solvate thereof, wherein R² is pyridyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Iaa), or a pharmaceutically acceptable salt or solvate thereof, wherein R² is pyridazinyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Iaa), or a pharmaceutically acceptable salt or solvate thereof, wherein R² is pyrimidinyl optionally substituted with one or more groups selected from halogen, C₁-C₆alkyl, C₁₋₆haloalkyl, —CN, —OR¹⁰, —N(R¹⁰)(R¹¹), and —S(O)₂R¹³. In some embodiments is a compound of Formula (Iaa), or a pharmaceutically acceptable salt or solvate thereof, wherein R² is pyrazinyl optionally substituted with one or more groups selected from halogen, C₁-C₆alkyl, C₁₋₆haloalkyl, —CN, —OR¹⁰, —N(R¹⁰)(R¹¹), and —S(O)₂R¹³.

In some embodiments is a compound of Formula (Iaa), or a pharmaceutically acceptable salt or solvate thereof, wherein R² is selected from unsubstituted pyridyl, unsubstituted pyridazinyl, unsubstituted pyrimidinyl, and unsubstituted pyrazinyl. In some embodiments is a compound of Formula (Iaa), or a pharmaceutically acceptable salt or solvate thereof, wherein R² is unsubstituted pyridyl. In some embodiments is a compound of Formula (Iaa), or a pharmaceutically acceptable salt or solvate thereof, wherein R² is unsubstituted pyridazinyl. In some embodiments is a compound of Formula (Iaa), or a pharmaceutically acceptable salt or solvate thereof, wherein R² is unsubstituted pyrimidinyl. In some embodiments is a compound of Formula (Iaa), or a pharmaceutically acceptable salt or solvate thereof, wherein R² is unsubstituted pyrazinyl.

In some embodiments is a compound of Formula (Iaa), or a pharmaceutically acceptable salt or solvate thereof, wherein R¹ and R² are combined to form a C₂-C₉heterocycloalkyl optionally substituted with one or more groups selected from halogen, C₁₋₆ alkyl, C₁₋₆haloalkyl, —OR¹⁰, —N(R¹⁰)(R¹¹), and —S(O)₂R¹³. In some embodiments is a compound of Formula (Iaa), or a pharmaceutically acceptable salt or solvate thereof, wherein R¹ and R² are combined to form a C₂-C₉heterocycloalkyl optionally substituted with one, two, or three groups selected from halogen, C₁₋₆alkyl, C₁₋₆haloalkyl, —OR¹⁰, —N(R¹⁰)(R¹¹), and —S(O)₂R¹³. In some embodiments is a compound of Formula (Iaa), or a pharmaceutically acceptable salt or solvate thereof, wherein R¹ and R² are combined to form an unsubstituted C₂-C₉heterocycloalkyl.

Any combination of the groups described above for the various variables is contemplated herein. Throughout the specification, groups and substituents thereof are chosen by one skilled in the field to provide stable moieties and compounds.

In some embodiments is a compound, or a pharmaceutically acceptable salt or solvate thereof, selected from:

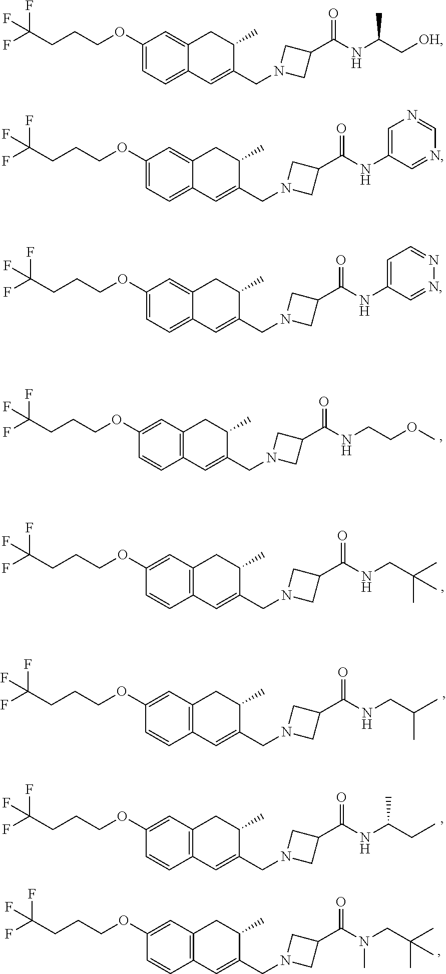

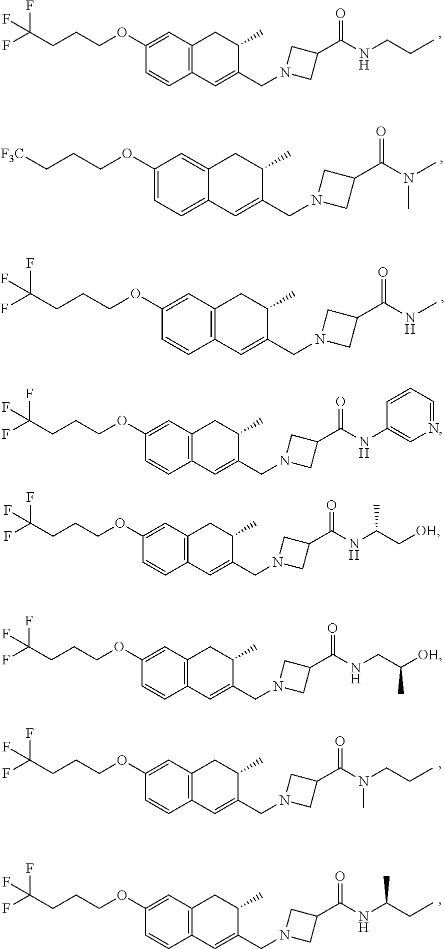

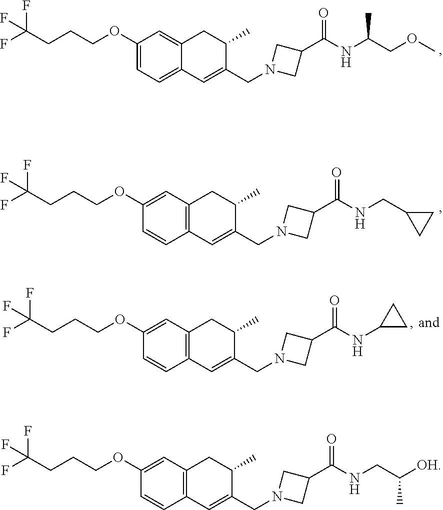 [SMILES: COC[C@H](C)NC(=O)C1CN(CC2=Cc3ccc(OCCCC(F)(F)F)cc3C[C@@H]2C)C1 ; C[C@H]1Cc2cc(OCCCC(F)(F)F)ccc2C=C1CN1CC(C(=O)NCC2CC2)C1 ; C[C@H]1Cc2cc(OCCCC(F)(F)F)ccc2C=C1CN1CC(C(=O)NC2CC2)C1 ; C[C@@H](O)CNC(=O)C1CN(CC2=Cc3ccc(OCCCC(F)(F)F)cc3C[C@@H]2C)C1]

In some embodiments is a compound, or a pharmaceutically acceptable salt or solvate thereof, selected from:

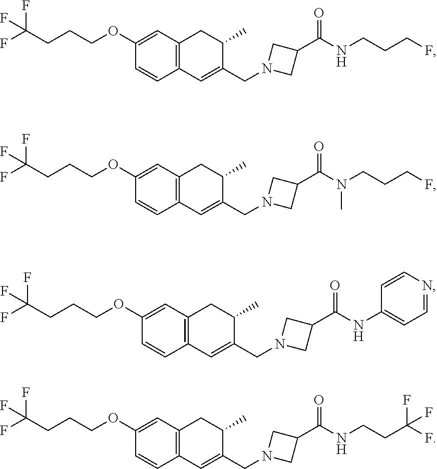 [SMILES: C[C@H]1Cc2cc(OCCCC(F)(F)F)ccc2C=C1CN1CC(C(=O)NCCCF)C1 ; C[C@H]1Cc2cc(OCCCC(F)(F)F)ccc2C=C1CN1CC(C(=O)N(C)CCCF)C1 ; C[C@H]1Cc2cc(OCCCC(F)(F)F)ccc2C=C1CN1CC(C(=O)Nc2ccncc2)C1 ; C[C@H]1Cc2cc(OCCCC(F)(F)F)ccc2C=C1CN1CC(C(=O)NCCC(F)(F)F)C1]

In some embodiments, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich: Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible, and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound described herein with an acid to provide a “pharmaceutically acceptable acid addition salt.” In some embodiments, the compound described herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; monomethyl fumarate, naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound described herein is prepared as a chloride salt, sulfate salt, bromide salt, mesylate salt, maleate salt, citrate salt or phosphate salt.

In some embodiments, pharmaceutically acceptable salts are obtained by reacting a compound described herein with a base to provide a “pharmaceutically acceptable base addition salt.”

In some embodiments, the compound described herein is acidic and is reacted with a base. In such situations, an acidic proton of the compound described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of isolating or purifying the compound with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), crystalline forms (also known as polymorphs), or pharmaceutically acceptable salts of compounds described herein, as well as active metabolites of these compounds having the same type of activity.

In some embodiments, sites on the organic radicals (e.g. alkyl groups, aromatic rings) of compounds described herein are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, one or more hydrogen atoms of the compounds described herein is replaced with deuterium.

In some embodiments, the compounds described herein possess one or more stereocenters and each stereocenter exists independently in either the R or S configuration. The compounds presented herein include all diastereomeric, enantiomeric, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley and Sons, Inc., 1981. In some embodiments, stereoisomers are obtained by stereoselective synthesis.

### Synthesis of Compounds

Compounds described herein are synthesized using standard synthetic techniques or using methods known in the art in combination with methods described herein.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions. The starting materials are available from commercial sources or are readily prepared.

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

In some embodiments, compounds are prepared as described in the Examples.

### Methods of Treatment

In some embodiments, described herein is a method of treating a CNS disease or disorder in a patient in need thereof comprising administering to the patient a compound of Formula (I), (Ia), or (Iaa), or a pharmaceutically acceptable salt or solvate thereof. In some embodiments is a method of treating a CNS disease or disorder in a patient in need thereof comprising administering to the patient a compound of Formula (I), (Ia), or (Iaa), or a pharmaceutically acceptable salt or solvate thereof, wherein the CNS disease or disorder is selected from acute disseminated encephalomyelitis (ADEM), acute hemorrhagic leukoencephalitis (AHL or AHLE), adult Refsum disease, infantile Refsum disease, Alexander disease, Alzheimer's disease, Balo concentric sclerosis, Canavan disease, central pontine myelinolysis (CPM), cerebral palsy, cerebrotendineous xanthomatosis, chronic inflammatory demyelinating polyneuropathy (CIDP), Devic's syndrome, diffuse myelinoclastic sclerosis, encephalomyelitis, Guillain-Barre syndrome, Huntington's disease (HD), idiopathic inflammatory demyelinating disease (HDD), Krabbe disease, Leber hereditary optic neuropathy, leukodystrophy, Marburg multiple sclerosis, Marchiafava-Bignami disease, metachromatic leukodystrophy (MLD), multifocal motor neuropathy (MMN), multiple sclerosis (MS), Neiman Pick Type-C, paraproteinemic demyelinating polyneuropathy, Parkinson's disease (PD), Pelizaeus-Merzbacher disease (PMD), progressive multifocal leukoencephalopathy (PML), tropical spastic paraparesis (TSP), X-linked adrenoleukodystrophy (X-ALD, ALO, or X-linked ALO), and Zellweger syndrome. In some embodiments is a method of treating a CNS disease or disorder in a patient in need thereof comprising administering to the patient a compound of Formula (I), (Ia), or (Iaa), or a pharmaceutically acceptable salt or solvate thereof, wherein the CNS disease or disorder is selected from multiple sclerosis. In some embodiments is a method of treating a CNS disease or disorder in a patient in need thereof comprising administering to the patient a compound of Formula (I), (Ia), or (Iaa), or a pharmaceutically acceptable salt or solvate thereof, wherein the CNS disease or disorder is X-linked adrenoleukodystrophy.

In some embodiments, described herein is a method of treating a CNS disease or disorder in a patient in need thereof comprising administering to the patient a compound of Formula (I), (Ia), or (Iaa), or a pharmaceutically acceptable salt or solvate thereof, further comprising administering to the patient a peripherally restricted fatty acid amide hydrolase (FAAH) inhibitor. In some embodiments is a method of treating a CNS disease or disorder in a patient in need thereof comprising administering to the patient a compound of Formula (I), (Ia), or (Iaa), or a pharmaceutically acceptable salt or solvate thereof, wherein the CNS disease or disorder is selected from acute disseminated encephalomyelitis (ADEM), acute hemorrhagic leukoencephalitis (AHL or AHLE), adult Refsum disease, infantile Refsum disease, Alexander disease, Alzheimer's disease, Balo concentric sclerosis, Canavan disease, central pontine myelinolysis (CPM), cerebral palsy, cerebrotendineous xanthomatosis, chronic inflammatory demyelinating polyneuropathy (CIDP), Devic's syndrome, diffuse myelinoclastic sclerosis, encephalomyelitis, Guillain-Barre syndrome, Huntington's disease (HD), idiopathic inflammatory demyelinating disease (HDD), Krabbe disease, Leber hereditary optic neuropathy, leukodystrophy, Marburg multiple sclerosis, Marchiafava-Bignami disease, metachromatic leukodystrophy (MLD), multifocal motor neuropathy (MMN), multiple sclerosis (MS), Neiman Pick Type-C, paraproteinemic demyelinating polyneuropathy, Parkinson's disease (PD), Pelizaeus-Merzbacher disease (PMD), progressive multifocal leukoencephalopathy (PML), tropical spastic paraparesis (TSP), X-linked adrenoleukodystrophy (X-ALD, ALO, or X-linked ALO), and Zellweger syndrome, further comprising administering to the patient a peripherally restricted fatty acid amide hydrolase (FAAH) inhibitor. In some embodiments is a method of treating a CNS disease or disorder in a patient in need thereof comprising administering to the patient a compound of Formula (I), (Ia), or (Iaa), or a pharmaceutically acceptable salt or solvate thereof, wherein the CNS disease or disorder is selected from multiple sclerosis, further comprising administering to the patient a peripherally restricted fatty acid amide hydrolase (FAAH) inhibitor. In some embodiments is a method of treating a CNS disease or disorder in a patient in need thereof comprising administering to the patient a compound of Formula (I), (Ia), or (Iaa), or a pharmaceutically acceptable salt or solvate thereof, wherein the CNS disease or disorder is X-linked adrenoleukodystrophy, further comprising administering to the patient a peripherally restricted fatty acid amide hydrolase (FAAH) inhibitor.

In some embodiments of the methods described herein, the peripherally restricted FAAH inhibitor is ASP-3652.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. In some embodiments disclosed herein, the pharmaceutical composition further comprises a peripherally restricted fatty acid amide hydrolase (FAAH) inhibitor. In some embodiments, the pharmaceutical composition further comprises a peripherally restricted FAAH inhibitor, wherein the peripherally restricted FAAH inhibitor is ASP-3652.

Administration of the compounds and compositions described herein can be affected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may also be formulated in rectal compositions such as suppositories or retention enemas, e.g., containing conventional suppository bases such as cocoa butter, polyethylene glycol, or other glycerides.

Pharmaceutical compositions may be administered topically, that is by non-systemic administration. This includes the application of a compound of the present invention externally to the epiderm is or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical compositions suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

In some embodiments, a compound disclosed herein is formulated in such a manner that delivery of the compound to a particular region of the gastrointestinal tract is achieved. For example, a compound disclosed herein is formulated for oral delivery with bioadhesive polymers, pH-sensitive coatings, time dependent, biodegradable polymers, microflora activated systems, and the like, in order to effect delivering of the compound to a particular region of the gastrointestinal tract.

In some embodiments, a compound disclosed herein is formulated to provide a controlled release of the compound. Controlled release refers to the release of the compound described herein from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

Approaches to deliver the intact therapeutic compound to the particular regions of the gastrointestinal tract (e.g. such as the colon), include:
(i) Coating with polymers: The intact molecule can be delivered to the colon without absorbing at the upper part of the intestine by coating of the drug molecule with the suitable polymers, which degrade only in the colon.
(ii) Coating with pH-sensitive polymers: The majority of enteric and colon targeted delivery systems are based on the coating of tablets or pellets, which are filled into conventional hard gelatin capsules. Most commonly used pH-dependent coating polymers are methacrylic acid copolymers, commonly known as Eudragit® S, more specifically Eudragit® L and Eudragit® S. Eudragit® L100 and S 100 are copolymers of methacrylic acid and methyl methacrylate.
(iii) Coating with biodegradable polymers;
(iv) Embedding in matrices;
(v) Embedding in biodegradable matrices and hydrogels;
(vi) Embedding in pH-sensitive matrices;
(vii) Timed release systems;
(viii)Redox-sensitive polymers;
(ix) Bioadhesive systems;
(x) Coating with microparticles;
(xi) Osmotic controlled drug delivery;

Another approach towards colon-targeted drug delivery or controlled-release systems includes embedding the drug in polymer matrices to trap it and release it in the colon. These matrices can be pH-sensitive or biodegradable. Matrix-Based Systems, such as multi-matrix (MMX)-based delayed-release tablets, ensure the drug release in the colon.

Additional pharmaceutical approaches to targeted delivery of therapeutics to particular regions of the gastrointestinal tract are known. Chourasia MK, Jain SK, Pharmaceutical approaches to colon targeted drug delivery systems., J Pharm Sci. 2003 January-Apr; 6(1):33-66. Patel M, Shah T, Amin A. Therapeutic opportunities in colon-specific drug-delivery systems Crit Rev Ther Drug Carrier Syst. 2007; 24(2):147-202. Kumar P, Mishra B. Colon targeted drug delivery systems—an overview. Curr Drug Deliv. 2008 July; 5(3):186-98. Van den Mooter G. Colon drug delivery. Expert Opin Drug Deliv. 2006 January; 3(1):111-25. Seth Amidon, Jack E. Brown, and Vivek S. Dave, Colon-Targeted Oral Drug Delivery Systems: Design Trends and Approaches, AAPS PharmSciTech. 2015 August; 16(4):731-741.

It should be understood that in addition to the ingredients particularly mentioned above, the compounds and compositions described herein may include other agents conventional in the art having regard to the type of formulation in question, for example those suitable for oral administration may include flavoring agents.

### Methods of Dosing and Treatment Regimens

In one embodiment, the compounds described herein, or a pharmaceutically acceptable salt thereof, are used in the preparation of medicaments for the treatment of diseases or conditions in a mammal that would benefit from administration of an SIP5 receptor agonist. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In some embodiments disclosed herein, are methods of administering an SIP5 receptor agonist in combination with an additional therapeutic agent. In some embodiments are methods of administering an S1P5 receptor agonist in combination with a peripherally restricted fatty acid amide hydrolase (FAAH) inhibitor. In some embodiments are methods of administering an S1P5 receptor agonist in combination with a peripherally restricted FAAH inhibitor, wherein the peripherally restricted FAAH inhibitor is ASP-3652.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments, wherein the patient's condition does not improve, upon the doctor's discretion, the compounds are administered chronically, that is, for an extended period of time, including throughout the duration of the patient's life in order to ameliorate or otherwise control or limit the symptoms of the patient's disease or condition.

In certain embodiments, wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In certain instances, it is appropriate to administer at least one compound described herein, or a pharmaceutically acceptable salt thereof, in combination with one or more other therapeutic agents.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound described herein, or a pharmaceutically acceptable salt thereof, is co-administered with a second therapeutic agent, wherein the compound described herein, or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient may be additive of the two therapeutic agents or the patient may experience a synergistic benefit.

In certain embodiments, different therapeutically-effective dosages of the compounds disclosed herein will be utilized in formulating pharmaceutical composition and/or in treatment regimens when the compounds disclosed herein are administered in combination with one or more additional agent, such as an additional therapeutically effective drug, an adjuvant or the like. Therapeutically-effective dosages of drugs and other agents for use in combination treatment regimens is optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt thereof, is initiated prior to, during, or after treatment with a second agent described herein, and continues until any time during treatment with the second agent or after termination of treatment with the second agent. It also includes treatments in which a compound described herein, or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

### Examples

The following examples are provided for illustrative purposes only and not to limit the scope of the claims provided herein.

As used above, and throughout the description of the invention, the following abbreviations, unless otherwise indicated, shall be understood to have the following meanings:
ACN or MeCN acetonitrile
AcOH acetic acid
Ac acetyl
BINAP 2,2′-bis(diphenylphosphino)-1,1′-binaphthalene
Bn benzyl
BOC or Boc tert-butyl carbamate
t-Bu tert-butyl
Cy cyclohexyl
DBA or dba dibenzylideneacetone
DCE dichloroethane (ClCH₂CH₂Cl)
DCM dichloromethane (CH₂Cl₂)
DIPEA or DIEA diisopropylethylamine
DMAP 4-(N,N-dimethylamino)pyridine
DME 1,2-dimethoxyethane
DMF N,N-dimethylformamide
DMA N,N-dimethylacetamide
DMSO dimethylsulfoxide
Dppf or dppf 1, l′-bis(diphenylphosphino) ferrocene
EEDQ 2-Ethoxy-1-ethoxycarbonyl-1,2-dihydroquinoline
eq equivalent(s)
Et ethyl
Et₂O diethyl ether
EtOH ethanol
EtOAc ethyl acetate
HATU 1-[bis(dimethylamino)methylene]-1H-1,2,3-triazolo[4,5-b]pyridinium 3-oxid hexafluorophosphate
HMPA hexamethylphosphoramide
HPLC high performance liquid chromatography
KHMDS potassium bis(trimethylsilyl)amide
NaHMDS sodium bis(trimethylsilyl)amide
LiHMDS lithium bis(trimethylsilyl)amide
LAH lithium aluminum anhydride
LCMS liquid chromatography mass spectrometry
Me methyl
MeOH methanol
MS mass spectroscopy
Ms mesyl
NBS N-bromosuccinimide
NMM N-methyl-morpholine
NMP N-methyl-pyrrolidin-2-one
NMR nuclear magnetic resonance
PCC pyridinium chlorochromate
Ph phenyl
PPTS pyridium p-toluenesulfonate
iPr/i-Pr iso-propyl
TBS tert-butyldimethylsilyl
RP-HPLC reverse phase-high pressure liquid chromatography
TFA trifluoroacetic acid
TEA triethylamine
THF tetrahydrofuran
TLC thin layer chromatography

### Example 1: Synthesis of (S)-1-((3-methyl-6-(4,4,4-trifluorobutoxy)-3,4-dihydronaphthalen-2-yl)methyl)azetidine-3-carboxylic acid (Compound 1)

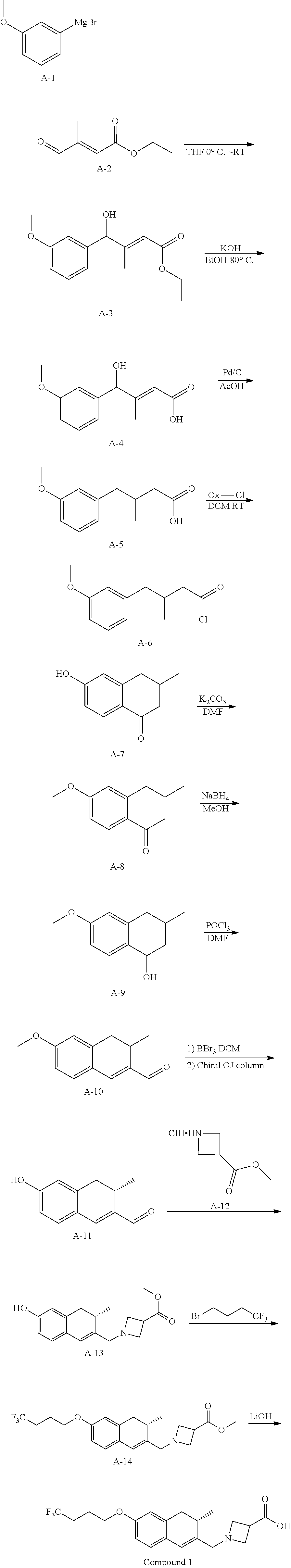

To a solution of A-2 (30.0 g, 211 mmol) in THF (300 mL) was added A-1 (44.6 g, 211 mmol) dropwise at 0° C. under N₂. The mixture was stirred at rt for 2 h. The reaction mixture was poured slowly into ice water (300 mL) and 6 N HCl (200 mL), and then extracted with EtOAc (200 mL*3). The combined organic phase was washed with brine (200 mL), dried over Na₂SO₄, and concentrated under reduce pressure. The residue was purified by flash column chromatography to afford A-3 (28.4 g, 54% yield) as a yellow oil.

To a solution of A-3 (28.4 g, 113 mmol) in ethanol (280 mL) and water (70 mL) was added KOH (15.9 g, 284 mmol), The mixture was stirred at 80° C. for 3 h. The reaction was adjusted to pH=2-3 with 6 N HCl. The mixture was diluted with water (100 mL) and extracted with DCM (200 mL*3). The combined organic phase was washed with brine (350 mL), dried over Na₂SO₄, and concentrated under reduce pressure to afford A-4 (22.2 g, 88% yield) as a red oil.

To a solution of A-4 (22.2 g, 99.9 mmol) in acetic acid (220 mL) was added 10% Pd/C (2.22 g). The mixture was stirred at rt for 16 h under H₂ balloon. The reaction was filtered and the filtration was concentrated. Water was added to the residue and the mixture was extracted with DCM. The organic layer were washed with water and brine, dried over Na₂SO₄, and then concentrated under reduce pressure to afford A-5 (16.0 g, 77% yield) as a gray oil.

To a solution of A-5 (16.0 g, 76.8 mmol) in DCM (160 mL) was added oxalyl chloride (48.8 g, 384.0 mmol) at rt. The mixture was stirred at rt for 15 h. The reaction mixture was concentrated under reduce pressure to afford A-6 (15.0 g, 86% yield) as a red oil.

To a solution of AlCl₃ (17.6 g, 132.4 mmol) in DCM (150 mL) was added A-6 (15.0 g, 66.2 mmol) in DCM dropwise at 0° C. The mixture was stirred at rt for 16 h. To the reaction mixture was added 6 N HCl (100 mL) and water (50 mL). The mixture was extracted with DCM (80 mL*3). The combined organic layers were washed with water, brine, dried over Na₂SO₄, and concentrated. The residue was purified by flash column chromatography to afford A-7 (8.6 g, 74% yield) as a yellow solid.

To a solution of A-7 (8.6 g, 48.9 mmol) in dried DMF (50 mL) was added CH₃I (10.4 g, 73.4 mmol) and K₂CO₃ (13.5 g, 97.8 mmol). The mixture was stirred at rt for 4 h. Water was added and the mixture was extracted with EtOAc. The organic layer was washed with water, brine, dried over Na₂SO₄, and then concentrated under reduced pressure. The residue was purified by flash column chromatography to afford A-8 (7.1 g, 76% yield) as a yellow solid.

To a solution of A-8 (7.1 g, 37.3 mmol) in MeOH (70 mL) was added NaBH₄ (2.82 g, 74.6 mmol) at 0° C. The mixture was stirred at rt for 4 h. Water was added and the mixture was extracted with DCM. The organic layer was washed with water, brine, dried over Na₂SO₄, and then concentrated under reduce pressure to afford A-9 (6.9 g, 96% yield) as a yellow oil.

To a solution of A-9 (6.9 g, 35.9 mmol) in DMF (70 mL) was added POCl₃ (16.5 g, 108.0 mmol) dropwise at rt. The mixture was stirred at 60° C. for 16 h. The reaction was poured into cold water (400 mL) and the mixture was stirred at rt for 10 mins. The mixture was extracted with EtOAc (100 mL*2). The combined organic layers were washed with water, brine, dried over Na₂SO₄, and then concentrated under reduce pressure. The residue was purified by flash column chromatography to afford A-10 (6.5 g, 90% yield) as a yellow oil.

To a solution of A-10 (6.5 g, 32.1 mmol) in DCM (70 mL) was added BBr₃ (17.7 g, 70.7 mmol) at 0° C. The mixture was stirred at 0° C. for 4 h. Water (100 mL) was added to the reaction and the mixture was extracted with DCM (80 mL*3). The combined organic layers were washed with water, brine, dried over Na₂SO₄, and concentrated. The residue was purified by flash column chromatography to afford racemic A-11 (4.5 g, 74.4% yield) as a brown solid. Racemic A-11 was purified by chiral separation (using HPLC(column used: Daicel Corporation CHIRALCEL OJ-H (4.6 mm I. D.×250 mm L), mobile phase: normal hexane: 2-propanol=80:20, flow rate: 1 mL/min, temperature: 40° C., wavelength: 245 nm), whereby A-11 (1.9 g) was obtained in the second peak (retention time: about 8.11 minutes) as a brown solid.

To a solution of A-12 (1.5 g, 10.1 mmol) in DCE (20 mL) was added DIEA (1.3 g, 10.1 mmol) at rt. After stirring for 10 mins, A-11 (1.9 g, 10.1 mmol) and AcOH (60.6 mg, 1.01 mmol) was added. The mixture was stirred at rt for 2 h. NaBH(OAc) 3 (4.28 g, 20.2 mmol) was added and the resulting mixture was stirred at rt for 16 h. Water was added and the mixture was extracted with DCM. The organic layer was washed with brine and concentrated. The residue was purified by flash column chromatography to afford A-13 (2.4 g, 83% yield) as a red oil.

To a solution of A-13 (2.4 g, 8.4 mmol) in MeCN(25 mL) was added 4-bromo-1,1,1-trifluorobutane (3.2 g, 16.7 mmol) and K₂CO₃ (2.3 g, 16.7 mmol) at rt. The mixture was stirred at 70° C. for 16 h. The reaction mixture was cooled to rt. Water (100 mL) was added to the reaction mixture and the mixture was extracted with EtOAc (30 ml*3). The combined organic phase was dried and concentrated. The residue was purified by flash column chromatography to afford A-15 (1.7 g, 51% yield) as a white solid.

To a solution of A-15 (1.7 g, 4.3 mmol) in THF (16 mL), was added LiOH·H₂O (361.0 mg, 8.6 mmol) and water (4 mL). The reaction mixture was stirred at rt for 2 hr. The mixture was adjusted pH=5~6 with 1N HCl and water (20 mL) was added. The mixture was extracted with EtOAc (20 mL*3). The combined organic phase was washed with water (30 mL) and brine (30 mL), dried over Na₂SO₄ and concentrated. The residue was purified by reverse phase column chromatography to afford Compound 1 (1.5 g, 91% yield) as a white solid. LCMS: 384.3 [M+1]⁺. Compound 1 is an SIP5 agonist.

### Example 2: Synthesis of N—((S)-1-hydroxypropan-2-yl)-1-(((S)-3-methyl-6-(4,4,4-trifluorobutoxy)-3,4-dihydronaphthalen-2-yl)methyl)azetidine-3-carboxamide (Compound 2)

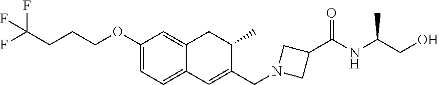 [SMILES: C[C@@H](CO)NC(=O)C1CN(CC2=Cc3ccc(OCCCC(F)(F)F)cc3C[C@@H]2C)C1]

A solution of Compound 1 (60 mg, 156 μmol), (S)-2-aminopropan-1-ol (14.1 mg, 188 μmol), HATU (89.3 mg, 235 μmol) and DIPEA (81.8 μL, 3 eq, 463 μmol) in DMF (2 mL) was stirred at room temperature for 16.0 h. Water (10 mL) was added, and the resulting mixture was extracted with EtOAc (10 mL*3). The combined organic phases were washed with water (10 mL*2), brine (10 mL), dried over Na₂SO₄, concentrated in vacuo and purified by prep-TLC to afford N—((S)-1-hydroxypropan-2-yl)-1-(((S)-3-methyl-6-(4,4,4-trifluorobutoxy)-3,4-dihydronaphthalen-2-yl)methyl)azetidine-3-carboxamide (Compound 2)(8 mg) as a white solid. MS(ESI): 441.3 (M+H).

### Example 3: Synthesis of (S)-1-((3-methyl-6-(4,4,4-trifluorobutoxy)-3,4-dihydronaphthalen-2-yl)methyl)-N-(pyrimidin-5-yl)azetidine-3-carboxamide (Compound 3)

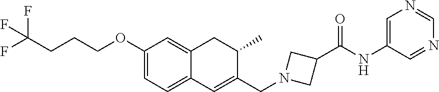 [SMILES: C[C@H]1Cc2cc(OCCCC(F)(F)F)ccc2C=C1CN1CC(C(=O)Nc2cncnc2)C1]

(S)-1-((3-methyl-6-(4,4,4-trifluorobutoxy)-3,4-dihydronaphthalen-2-yl)methyl)-N-(pyrimidin-5-yl)azetidine-3-carboxamide (Compound 3) was prepared as described in Example 2 starting from Compound 1 and pyrimidin-5-amine. MS(ESI): 461.3 (M+H).

### Example 4: Synthesis of (S)-1-((3-methyl-6-(4,4,4-trifluorobutoxy)-3,4-dihydronaphthalen-2-yl)methyl)-N-(pyridazin-4-yl)azetidine-3-carboxamide (Compound 4)

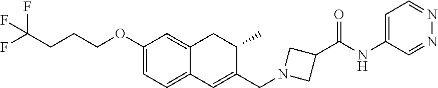 [SMILES: C[C@H]1Cc2cc(OCCCC(F)(F)F)ccc2C=C1CN1CC(C(=O)Nc2ccnnc2)C1]

(S)-1-((3-methyl-6-(4,4,4-trifluorobutoxy)-3,4-dihydronaphthalen-2-yl)methyl)-N-(pyridazin-4-yl)azetidine-3-carboxamide (Compound 4) was prepared as described in Example 2 starting from Compound 1 and pyridazin-4-amine. MS(ESI): 461.3 (M+H).

### Example 5: Synthesis of (S)—N-(2-methoxyethyl)-1-((3-methyl-6-(4,4,4-trifluorobutoxy)-3,4-dihydronaphthalen-2-yl)methyl)azetidine-3-carboxamide (Compound 5)

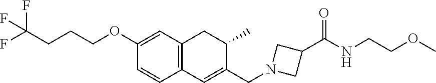 [SMILES: COCCNC(=O)C1CN(CC2=Cc3ccc(OCCCC(F)(F)F)cc3C[C@@H]2C)C1]

(S)—N-(2-methoxyethyl)-1-((3-methyl-6-(4,4,4-trifluorobutoxy)-3,4-dihydronaphthalen-2-yl)methyl)azetidine-3-carboxamide (Compound 5) was prepared as described in Example 2 starting from Compound 1 and 2-methoxyethan-1-amine. MS(ESI): 441.3 (M+H).

### Example 6: Synthesis of (S)-1-((3-methyl-6-(4,4,4-trifluorobutoxy)-3,4-dihydronaphthalen-2-yl)methyl)-N-neopentylazetidine-3-carboxamide (Compound 6)

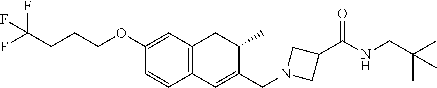 [SMILES: C[C@H]1Cc2cc(OCCCC(F)(F)F)ccc2C=C1CN1CC(C(=O)NCC(C)(C)C)C1]

(S)-1-((3-methyl-6-(4,4,4-trifluorobutoxy)-3,4-dihydronaphthalen-2-yl)methyl)-N-neopentylazetidine-3-carboxamide (Compound 6) was prepared as described in Example 2 starting from Compound 1 and 2,2-dimethylpropan-1-amine. MS(ESI): 453.3 (M+H).

### Example 7: Synthesis of (S)—N-isobutyl-1-((3-methyl-6-(4,4,4-trifluorobutoxy)-3,4-dihydronaphthalen-2-yl)methyl)azetidine-3-carboxamide (Compound 7)

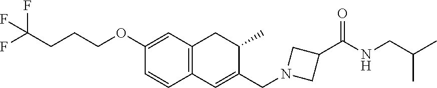 [SMILES: CC(C)CNC(=O)C1CN(CC2=Cc3ccc(OCCCC(F)(F)F)cc3C[C@@H]2C)C1]

(S)—N-isobutyl-1-((3-methyl-6-(4,4,4-trifluorobutoxy)-3,4-dihydronaphthalen-2-yl)methyl)azetidine-3-carboxamide (Compound 7) was prepared as described in Example 2 starting from Compound 1 and 2-methylpropan-1-amine. MS(ESI): 439.3 (M+H).

### Example 8: Synthesis of N—((R)-sec-butyl)-1-(((S)-3-methyl-6-(4,4,4-trifluorobutoxy)-3,4-dihydronaphthalen-2-yl)methyl)azetidine-3-carboxamide (Compound 8)

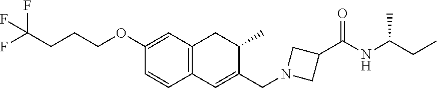 [SMILES: CC[C@@H](C)NC(=O)C1CN(CC2=Cc3ccc(OCCCC(F)(F)F)cc3C[C@@H]2C)C1]

N—((R)-sec-butyl)-1-(((S)-3-methyl-6-(4,4,4-trifluorobutoxy)-3,4-dihydronaphthalen-2-yl)methyl)azetidine-3-carboxamide (Compound 8) was prepared as described in Example 2 starting from Compound 1 and (2R)-butan-2-amine. MS(ESI): 439.3 (M+H).

### Example 9: Synthesis of (S)—N-methyl-1-((3-methyl-6-(4,4,4-trifluorobutoxy)-3,4-dihydronaphthalen-2-yl)methyl)-N-neopentylazetidine-3-carboxamide (Compound 9)

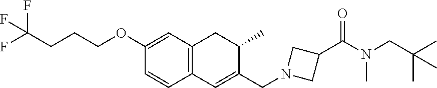 [SMILES: C[C@H]1Cc2cc(OCCCC(F)(F)F)ccc2C=C1CN1CC(C(=O)N(C)CC(C)(C)C)C1]

(S)—N-methyl-1-((3-methyl-6-(4,4,4-trifluorobutoxy)-3,4-dihydronaphthalen-2-yl)methyl)-N-neopentylazetidine-3-carboxamide (Compound 9) was prepared as described in Example 2 starting from Compound 1 and (2,2-dimethylpropyl)(methyl)amine. MS(ESI): 467.3 (M+H).

### Example 10: Synthesis of (S)-1-((3-methyl-6-(4,4,4-trifluorobutoxy)-3,4-dihydronaphthalen-2-yl)methyl)-N-propylazetidine-3-carboxamide (Compound 10)

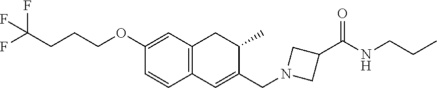 [SMILES: CCCNC(=O)C1CN(CC2=Cc3ccc(OCCCC(F)(F)F)cc3C[C@@H]2C)C1]

(S)-1-((3-methyl-6-(4,4,4-trifluorobutoxy)-3,4-dihydronaphthalen-2-yl)methyl)-N-propylazetidine-3-carboxamide (Compound 10) was prepared as described in Example 2 starting from Compound 1 and propan-1-amine. MS(ESI): 425.4 (M+H).

### Example 11: Synthesis of (S)—N,N-dimethyl-1-((3-methyl-6-(4,4,4-trifluorobutoxy)-3,4-dihydronaphthalen-2-yl)methyl)azetidine-3-carboxamide (Compound 11)

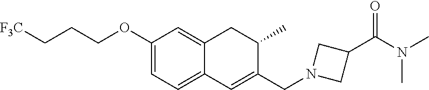 [SMILES: C[C@H]1Cc2cc(OCCCC(F)(F)F)ccc2C=C1CN1CC(C(=O)N(C)C)C1]

(S)—N,N-dimethyl-1-((3-methyl-6-(4,4,4-trifluorobutoxy)-3,4-dihydronaphthalen-2-yl)methyl)azetidine-3-carboxamide (Compound 11) was prepared as described in Example 2 starting from Compound 1 and dimethylamine. MS(ESI): 411.2 (M+H).

### Example 12: Synthesis of (S)—N-methyl-1-((3-methyl-6-(4,4,4-trifluorobutoxy)-3,4-dihydronaphthalen-2-yl)methyl)azetidine-3-carboxamide (Compound 12)

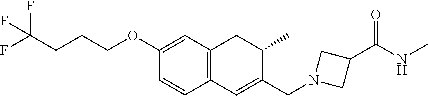 [SMILES: CNC(=O)C1CN(CC2=Cc3ccc(OCCCC(F)(F)F)cc3C[C@@H]2C)C1]

(S)—N-methyl-1-((3-methyl-6-(4,4,4-trifluorobutoxy)-3,4-dihydronaphthalen-2-yl)methyl)azetidine-3-carboxamide (Compound 12) was prepared as described in Example 2 starting from Compound 1 and methylamine. MS(ESI): 397.3 (M+H).

### Example 13: Synthesis of (S)-1-((3-methyl-6-(4,4,4-trifluorobutoxy)-3,4-dihydronaphthalen-2-yl)methyl)-N-(pyridin-3-yl)azetidine-3-carboxamide (Compound 13)

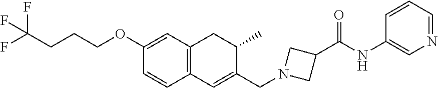 [SMILES: C[C@H]1Cc2cc(OCCCC(F)(F)F)ccc2C=C1CN1CC(C(=O)Nc2cccnc2)C1]

(S)-1-((3-methyl-6-(4,4,4-trifluorobutoxy)-3,4-dihydronaphthalen-2-yl)methyl)-N-(pyridin-3-yl)azetidine-3-carboxamide (Compound 13) was prepared as described in Example 2 starting from Compound 1 and pyridin-3-amine. MS(ESI): 460.2 (M+H).

### Example 14: Synthesis of N—((R)-1-hydroxypropan-2-yl)-1-(((S)-3-methyl-6-(4,4,4-trifluorobutoxy)-3,4-dihydronaphthalen-2-yl)methyl)azetidine-3-carboxamide (Compound 14)

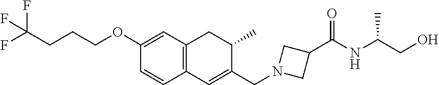 [SMILES: C[C@H](CO)NC(=O)C1CN(CC2=Cc3ccc(OCCCC(F)(F)F)cc3C[C@@H]2C)C1]

N—((R)-1-hydroxypropan-2-yl)-1-(((S)-3-methyl-6-(4,4,4-trifluorobutoxy)-3,4-dihydronaphthalen-2-yl)methyl)azetidine-3-carboxamide (Compound 14) was prepared as described in Example 2 starting from Compound 1 and (R)-2-aminopropan-1-ol. MS(ESI): 441.3 (M+H).

### Example 15: Synthesis of N—((S)-2-hydroxypropyl)-1-(((S)-3-methyl-6-(4,4,4-trifluorobutoxy)-3,4-dihydronaphthalen-2-yl)methyl)azetidine-3-carboxamide (Compound 15)

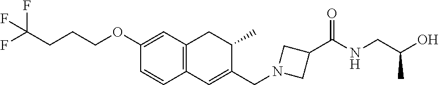 [SMILES: C[C@H](O)CNC(=O)C1CN(CC2=Cc3ccc(OCCCC(F)(F)F)cc3C[C@@H]2C)C1]

N—((S)-2-hydroxypropyl)-1-(((S)-3-methyl-6-(4,4,4-trifluorobutoxy)-3,4-dihydronaphthalen-2-yl)methyl)azetidine-3-carboxamide (Compound 15) was prepared as described in Example 2 starting from Compound 1 and(S)-1-aminopropan-2-ol. MS(ESI): 441.3 (M+H).

### Example 16: Synthesis of (S)—N-methyl-1-((3-methyl-6-(4,4,4-trifluorobutoxy)-3,4-dihydronaphthalen-2-yl)methyl)-N-propylazetidine-3-carboxamide (Compound 16)

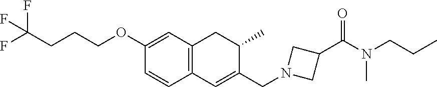 [SMILES: CCCN(C)C(=O)C1CN(CC2=Cc3ccc(OCCCC(F)(F)F)cc3C[C@@H]2C)C1]

(S)—N-methyl-1-((3-methyl-6-(4,4,4-trifluorobutoxy)-3,4-dihydronaphthalen-2-yl)methyl)-N-propylazetidine-3-carboxamide (Compound 16) was prepared as described in Example 2 starting from Compound 1 and N-methylpropan-1-amine. MS(ESI): 439.3 (M+H).

### Example 17: Synthesis of N—((S)-sec-butyl)-1-(((S)-3-methyl-6-(4,4,4-trifluorobutoxy)-3,4-dihydronaphthalen-2-yl)methyl)azetidine-3-carboxamide (Compound 17)

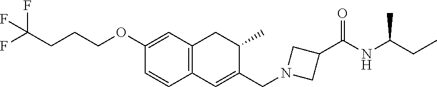 [SMILES: CC[C@H](C)NC(=O)C1CN(CC2=Cc3ccc(OCCCC(F)(F)F)cc3C[C@@H]2C)C1]

N—((S)-sec-butyl)-1-(((S)-3-methyl-6-(4,4,4-trifluorobutoxy)-3,4-dihydronaphthalen-2-yl)methyl)azetidine-3-carboxamide (Compound 17) was prepared as described in Example 2 starting from Compound 1 and(S)-butan-2-amine. MS(ESI): 439.3 (M+H).

### Example 18: Synthesis of N—((S)-1-methoxypropan-2-yl)-1-(((S)-3-methyl-6-(4,4,4-trifluorobutoxy)-3,4-dihydronaphthalen-2-yl)methyl)azetidine-3-carboxamide (Compound 18)

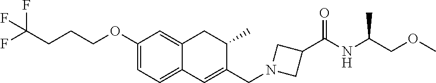 [SMILES: COC[C@H](C)NC(=O)C1CN(CC2=Cc3ccc(OCCCC(F)(F)F)cc3C[C@@H]2C)C1]

N—((S)-1-methoxypropan-2-yl)-1-(((S)-3-methyl-6-(4,4,4-trifluorobutoxy)-3,4-dihydronaphthalen-2-yl)methyl)azetidine-3-carboxamide (Compound 18) was prepared as described in Example 2 starting from Compound 1 and 1-methoxypropan-2-amine. MS(ESI): 455.3 (M+H).

### Example 19: Synthesis of (S)—N-(cyclopropylmethyl)-1-((3-methyl-6-(4,4,4-trifluorobutoxy)-3,4-dihydronaphthalen-2-yl)methyl)azetidine-3-carboxamide (Compound 19)

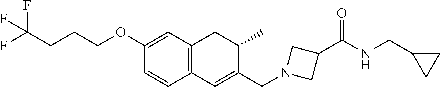 [SMILES: C[C@H]1Cc2cc(OCCCC(F)(F)F)ccc2C=C1CN1CC(C(=O)NCC2CC2)C1]

(S)—N-(cyclopropylmethyl)-1-((3-methyl-6-(4,4,4-trifluorobutoxy)-3,4-dihydronaphthalen-2-yl)methyl)azetidine-3-carboxamide (Compound 19) was prepared as described in Example 2 starting from Compound 1 and cyclopropylmethanamine. MS(ESI): 437.3 (M+H).

### Example 20: Synthesis of (S)—N-cyclopropyl-1-((3-methyl-6-(4,4,4-trifluorobutoxy)-3,4-dihydronaphthalen-2-yl)methyl)azetidine-3-carboxamide (Compound 20)

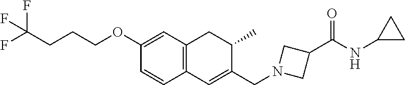 [SMILES: C[C@H]1Cc2cc(OCCCC(F)(F)F)ccc2C=C1CN1CC(C(=O)NC2CC2)C1]

(S)—N-cyclopropyl-1-((3-methyl-6-(4,4,4-trifluorobutoxy)-3,4-dihydronaphthalen-2-yl)methyl)azetidine-3-carboxamide (Compound 20) was prepared as described in Example 2 starting from Compound 1 and cyclopropanamine. MS(ESI): 423.3 (M+H).

### Example 21: Synthesis of N—((R)-2-hydroxypropyl)-1-(((S)-3-methyl-6-(4,4,4-trifluorobutoxy)-3,4-dihydronaphthalen-2-yl)methyl)azetidine-3-carboxamide (Compound 21)

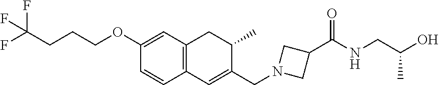 [SMILES: C[C@@H](O)CNC(=O)C1CN(CC2=Cc3ccc(OCCCC(F)(F)F)cc3C[C@@H]2C)C1]

N—((R)-2-hydroxypropyl)-1-(((S)-3-methyl-6-(4,4,4-trifluorobutoxy)-3,4-dihydronaphthalen-2-yl)methyl)azetidine-3-carboxamide (Compound 21) was prepared as described in Example 2 starting from Compound 1 and (R)-1-aminopropan-2-ol. MS(ESI): 441.3 (M+H).

Compounds 22-25 were prepared using similar procedures as described in the preceding examples.

| Cmpd # | Structure | Name | [M + H]⁺ |
|---|---|---|---|
| 22 | 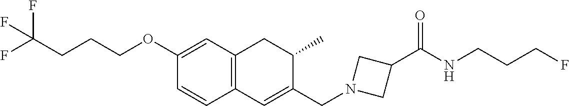 [SMILES: C[C@H]1Cc2cc(OCCCC(F)(F)F)ccc2C=C1CN1CC(C(=O)NCCCF)C1] | (S)-N-(3- fluoropropyl)-1-((3- methyl-6-(4,4,4- trifluorobutoxy)- 3,4-dihydro- naphthalen-2- yl)methyl)azetidine- 3-carboxamide | 443.1 |
| 23 | 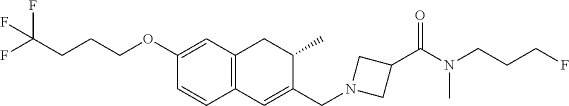 [SMILES: C[C@H]1Cc2cc(OCCCC(F)(F)F)ccc2C=C1CN1CC(C(=O)N(C)CCCF)C1] | (S)-N-(3-fluoropropyl)- N-methyl-1- ((3-methyl-6-(4,4,4- trifluorobutoxy)-3,4- dihydronaphthalen-2- yl)methyl)azetidine- 3-carboxamide | 457.1 |
| 24 | 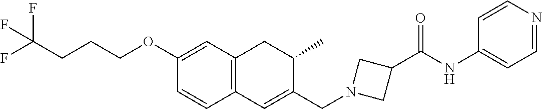 [SMILES: C[C@H]1Cc2cc(OCCCC(F)(F)F)ccc2C=C1CN1CC(C(=O)Nc2ccncc2)C1] | (S)-1-((3- methyl-6-(4,4,4- trifluorobutoxy)-3,4- dihydronaphthalen- 2-yl)methyl)-N- (pyridin-4-yl) azetidine-3- carboxamide | 460.0 |
| 25 | 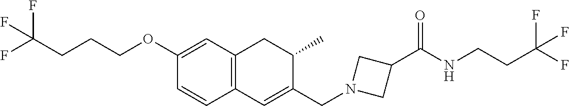 [SMILES: C[C@H]1Cc2cc(OCCCC(F)(F)F)ccc2C=C1CN1CC(C(=O)NCCC(F)(F)F)C1] | (S)-1-((3-methyl- 6-(4,4,4- trifluorobutoxy)-3,4- dihydronaphthalen-2-yl) methyl)-N- (3,3,3-trifluoropropyl) azetidine-3- carboxamide | 479.0 |

### Example 22: FAAH Substrate Evaluation

Purified recombinant human FAAH (rhFAAH) was purchased from Cayman Chemical (Ann Arbor, MI, USA). The total volume for each incubation was 400 μL containing a final 0.5 ng/μL rhFAAH, 1 μM test compound, 1.25% ethanol or 1 μM PF-3845 (FAAH inhibitor), and 0.1% bovine serum albumin in Tris-EDTA buffer at pH 8.0). The positive control was LL-341001. The incubation was conducted at the room temperature. At 0, 5, 15, 30 and 60 minutes, an aliquot of 30 μL reaction mixtures was removed and mixed with 300 μL acetonitrile containing 5 ng/mL terfenadine and 10 ng/ml tolbutamide as internal standards to quench the reaction. The resulting mixture was centrifuged at 4000 rpm, 4° C. for 15 minutes, and 100 μL supernatant was ready for LC-MS/MS analysis to measure the formation of acid metabolite.

### LC-MS/MS Analysis

Acquity Ultra Performance LC system from Waters was used for sample analysis. The chromatography was performed on a reverse phase Kinetex 2.6 μm C18 column, 2.1×30 mm, 100 Å. The mobile phase A comprised of 0.1% formic acid in water and mobile phase B comprised of 0.1% formic acid in acetonitrile with a 2-min run time at the flow rate of 0.8 mL/min for the acid metabolite from positive control or a 1.5 min run time at the flow rate of 0.9 mL/min for the acid metabolite of test compounds. The mass spectrometer (API-5500 and API Q Trap 4000 Applied Biosystems/MDS SCIEX Instruments, Framingham, MA, USA) was operated under ESI positive or negative ion MRM mode.

### Data Analysis

The formation of acid metabolite was monitored and quantified using one calibration point of 1 μM. The observed rate constant (ke) for the acid metabolite formation was calculated by plotting the metabolite concentration versus time of incubation with the slope being ke and is shown in Table 1.

TABLE 1

| Compound | ke | Compound | ke | Compound | ke |
|---|---|---|---|---|---|
| 2 | B | 3 | A | 4 | A |
| 5 | A | 6 | B | 7 | A |
| 8 | A | 9 | C | 10 | A |
| 11 | B | 12 | B | 13 | A |
| 14 | B | 15 | B | 16 | C |
| 17 | B | 18 | B | 19 | A |
| 20 | A | 21 | B | 22 | B |
| 23 | NT | 24 | A | 25 | A |

where A = ke is more than or equal to 1.0;
B = ke is less than 1.0 and more than 0;
C = ke is 0;
NT = not tested

The examples and embodiments described herein are for illustrative purposes only and various modifications or changes suggested to persons skilled in the art are to be included within the spirit and purview of this application and scope of the appended claims.

What is claimed is:

1. A compound of Formula (I), or a pharmaceutically acceptable salt or solvate thereof: 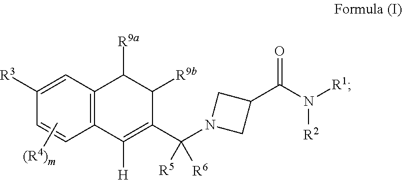
wherein:
R¹ is selected from hydrogen, —OR⁷, —NR⁷R⁸, C₁-C₆alkyl, C₂-C₆lkenyl, C₂-C₆alkynyl, C₃-C₆cycloalkyl, C₂-C₆heterocycloalkyl, C₁₋₉heteroaryl, phenyl, and —C₁-C₆alkyl-phenyl, wherein C₁-C₆alkyl, C₂-C₆alkenyl, C₂-C₆alkynyl, C₃-C₆cycloalkyl, C₂-C₉heterocycloalkyl, C₁₋₉heteroaryl, phenyl, and —C₁-C₆alkyl-phenyl are optionally substituted with one or more groups selected from halogen, C₁-C₆alkyl, C₁₋₆ haloalkyl, C₃-C₆cycloalkyl, —CN, —OR¹⁰, —N(R¹⁰)(R¹¹), and —S(O)₂R¹³;
R² is selected from hydrogen, —OR⁷, —NR⁷R⁸, C₁-C₆alkyl, C₂-C₆alkenyl, C₂-C₆alkynyl, C₃-C₆cycloalkyl, C₂-C₆heterocycloalkyl, C₁₋₉heteroaryl, phenyl, and —C₁-C₆alkyl-phenyl, wherein C₁-C₆alkyl, C₂-C₆alkenyl, C₂-C₆alkynyl, C₃-C₆cycloalkyl, C₂-C₉heterocycloalkyl, C₁₋₉heteroaryl, phenyl, and —C₁-C₆alkyl-phenyl are optionally substituted with one or more groups selected from halogen, C₁-C₆alkyl, C₁₋₆ haloalkyl, C₃-C₆cycloalkyl, —CN, —OR¹⁰, —N(R¹⁰)(R¹¹), and —S(O)₂R¹³; or R¹ and R² are combined to form a C₂-C₉heterocycloalkyl optionally substituted with one or more groups selected from halogen, C₁₋₆alkyl, C₁₋₆haloalkyl, —OR¹⁰, —N(R¹⁰)(R¹¹), and —S(O)₂R¹³;
R³ is selected from halogen, —CN, C₁₋₆alkyl, C₁₋₆haloalkyl, C₂₋₆alkenyl, C₂₋₆alkynyl, —OR¹⁰, —SR¹⁰, —N(R¹⁰)(R¹¹), —C(O)R¹³, —S(O)₂R¹³, and —S(O)₂N(R¹⁰)(R¹¹)—, wherein C₁₋₆ alkyl, C₂₋₆alkenyl, and C₂₋₆alkynyl, are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R⁴ is independently selected from halogen, —CN, C₁₋₆alkyl, C₁₋₆haloalkyl, C₂₋₆ alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²) C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(0)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰,
R⁵ and R⁶ are independently selected from hydrogen and C₁-C₆alkyl;
each R⁷ and each R⁸ are independently selected from hydrogen and C₁-C₆alkyl;
R^{9a} and R^{9b} are independently selected from hydrogen, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), wherein C₁₋₆alkyl, C₂₋₆alkenyl, C₂₋₆ alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, —C₁₋₆alkyl-C₃₋₆cycloalkyl, C₂₋₉heterocycloalkyl, —C₁₋₆ alkyl-C₂₋₉heterocycloalkyl, C₆₋₁₀aryl, —C₁₋₆alkyl-C₆₋₁₀aryl, C₁₋₉heteroaryl, and —C₁₋₆ alkyl-C₁₋₉heteroaryl, wherein C₁₋₆alkyl, C₂₋₆alkenyl, C₂₋₆alkynyl, C₃₋₆cycloalkyl, —C₁₋₆ alkyl-C₃₋₆cycloalkyl, C₂₋₉heterocycloalkyl, —C₁₋₆alkyl-C₂₋₉heterocycloalkyl, C₆₋₁₀aryl, —C₁₋₆alkyl-C₆₋₁₀aryl, C₁₋₉heteroaryl, and —C₁₋₆alkyl-C₁₋₉heteroaryl are optionally substituted with one, two, or three groups selected from halogen, —CN, hydroxy, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉ heterocycloalkyl, C₆₋₁₀aryl, and C₁₋₉heteroaryl, wherein C₁₋₆alkyl, C₂₋₆alkenyl, C₂₋₆ alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
m is 0, 1, 2, or 3.

2. The compound of claim 1, or a pharmaceutically acceptable salt or solvate thereof, wherein R⁵ and R⁶ are hydrogen.

3. The compound of claim 1 or claim 2, or a pharmaceutically acceptable salt or solvate thereof, wherein R^{9a} and R^{9b} are independently selected from hydrogen and C₁₋₆alkyl.

4. The compound of any one of claims 1-3, or a pharmaceutically acceptable salt or solvate thereof, wherein R^{9a} is hydrogen and R^{9b} is C₁₋₆alkyl.

5. The compound of any one of claims 1-4, or a pharmaceutically acceptable salt or solvate thereof, wherein R^{9a} is hydrogen and R^{9b} is —CH₃.

6. The compound of any one of claims 1-5, or a pharmaceutically acceptable salt or solvate thereof, wherein R³ is selected from C₁₋₆alkyl, C₁₋₆haloalkyl, —OR¹⁰, —SR¹⁰, —N(R¹⁰)(R¹¹), and —C(O)R¹³, wherein C₁₋₆alkyl is optionally substituted with one, two, or three groups selected from halogen, C₁₋₆haloalkyl, —OR¹⁰, and —N(R¹⁰)(R¹¹).

7. The compound of any one of claims 1-6, or a pharmaceutically acceptable salt or solvate thereof, wherein R³ is selected from C₁₋₆alkyl, C₁₋₆haloalkyl, —OR¹⁰, —SR¹⁰, and —N(R¹⁰)(R¹¹), wherein C₁₋₆alkyl is optionally substituted with one group selected from —OR¹⁰ and —N(R¹⁰)(R¹¹).

8. The compound of any one of claims 1-7, or a pharmaceutically acceptable salt or solvate thereof, wherein R³ is selected from —OR¹⁰, —SR¹⁰, and —N(R¹⁰)(R¹¹), wherein R¹⁰ and R¹¹ are independently selected from hydrogen, C₁₋₆alkyl, and C₁₋₆haloalkyl.

9. The compound of any one of claims 1-8, or a pharmaceutically acceptable salt or solvate thereof, wherein R³ is selected from —O(CH₂)₃CF³, —O(CH₂)₄CF³, —O(CH₂)₅CF³, —S(CH₂)₃CF³, —S(CH₂)₄CF³, —S(CH₂)₅CF³, —N(H)(CH₂)₃CF³, —N(H)(CH₂)₄CF³, and —N(H)(CH₂)₅CF³.

10. The compound of any one of claims 1-9, or a pharmaceutically acceptable salt or solvate thereof, wherein m is 0.

11. The compound of any one of claims 1-10, or a pharmaceutically acceptable salt or solvate thereof, having the structure of Formula (Ia):

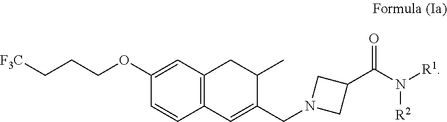

12. The compound of any one of claims 1-11, or a pharmaceutically acceptable salt or solvate thereof, having the structure of Formula (Iaa):

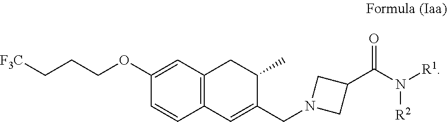

13. The compound of any one of claims 1-12, or a pharmaceutically acceptable salt or solvate thereof, wherein R¹ is hydrogen.

14. The compound of any one of claims 1-12, or a pharmaceutically acceptable salt or solvate thereof, wherein R¹ is C₁-C₆alkyl.

15. The compound of any one of claims 1-14, or a pharmaceutically acceptable salt or solvate thereof, wherein R² is C₁-C₆alkyl optionally substituted with one or more groups selected from halogen, C₁-C₆alkyl, C₁₋₆haloalkyl, —CN, —OR¹⁰, —N(R¹⁰)(R¹¹), and —S(O)₂R¹³.

16. The compound of any one of claims 1-15, or a pharmaceutically acceptable salt or solvate thereof, wherein R² is C₁-C₆alkyl substituted with one or more groups selected from halogen, C₁₋₆haloalkyl, —OR¹⁰, and —N(R¹⁰)(R¹¹).

17. The compound of any one of claims 1-16, or a pharmaceutically acceptable salt or solvate thereof, wherein R² is C₁-C₆alkyl substituted with one or more-OH.

18. The compound of any one of claims 1-17, or a pharmaceutically acceptable salt or solvate thereof, wherein R² is C₁-C₆alkyl substituted with one or more of halogen.

19. The compound of any one of claims 1-15, or a pharmaceutically acceptable salt or solvate thereof, wherein R² is unsubstituted C₁-C₆alkyl.

20. The compound of claim 19, or a pharmaceutically acceptable salt or solvate thereof, wherein R² is —CH₃.

21. The compound of any one of claims 1-14, or a pharmaceutically acceptable salt or solvate thereof, wherein R² is C₃-C₆cycloalkyl optionally substituted with one or more groups selected from halogen, C₁-C₆alkyl, C₁₋₆haloalkyl, —OR¹⁰, and —N(R¹⁰)(R¹¹).

22. The compound of claim 21, or a pharmaceutically acceptable salt or solvate thereof, wherein R² is unsubstituted C₃-C₆cycloalkyl.

23. The compound of any one of claims 1-14, or a pharmaceutically acceptable salt or solvate thereof, wherein R² is C₁₋₉heteroaryl optionally substituted with one or more groups selected from halogen, C₁-C₆alkyl, C₁₋₆haloalkyl, —CN, —OR¹⁰, —N(R¹⁰)(R¹¹), and —S(O)₂R¹³.

24. The compound of claim 23, or a pharmaceutically acceptable salt or solvate thereof, wherein R² is C₁₋₉heteroaryl optionally substituted with one or more groups selected from halogen, C₁-C₆alkyl, C₁₋₆haloalkyl, —CN, —OR¹⁰, —N(R¹⁰)(R¹¹), and —S(O)₂R¹³, wherein C₁₋₉heteroaryl is selected from pyridyl, pyridazinyl, pyrimidinyl, and pyrazinyl.

25. The compound of claim 24, or a pharmaceutically acceptable salt or solvate thereof, wherein R² is selected from unsubstituted pyridyl, unsubstituted pyridazinyl, unsubstituted pyrimidinyl, and unsubstituted pyrazinyl.

26. The compound of any one of claims 1-14, or a pharmaceutically acceptable salt or solvate thereof, wherein R² is hydrogen.

27. The compound of any one of claims 1-12, or a pharmaceutically acceptable salt or solvate thereof, wherein R¹ and R² are combined to form a C₂-C₉heterocycloalkyl optionally substituted with one or more groups selected from halogen, C₁₋₆alkyl, C₁₋₆ haloalkyl, —OR¹⁰, —N(R¹⁰)(R¹¹), and —S(O)₂R¹³.

28. The compound of claim 27, or a pharmaceutically acceptable salt or solvate thereof, wherein R¹ and R² are combined to form an unsubstituted C₂-C₆heterocycloalkyl.

29. A compound selected from: 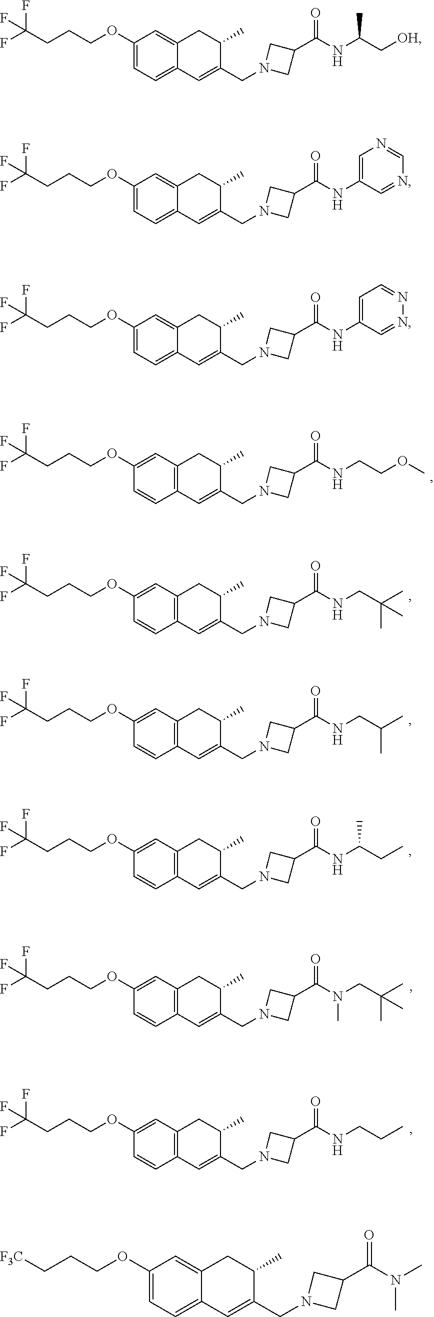 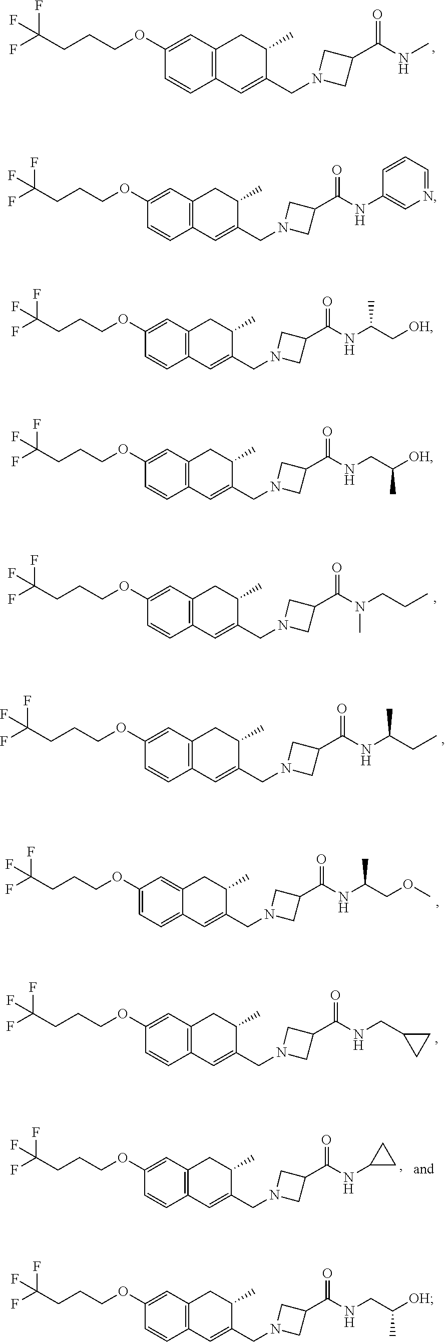
or a pharmaceutically acceptable salt or solvate thereof.

30. A compound selected from: 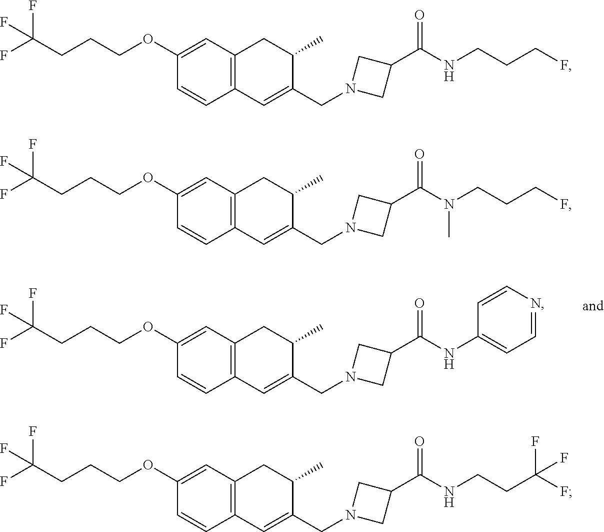 [SMILES: C[C@H]1Cc2cc(OCCCC(F)(F)F)ccc2C=C1CN1CC(C(=O)NCCCF)C1 ; C[C@H]1Cc2cc(OCCCC(F)(F)F)ccc2C=C1CN1CC(C(=O)N(C)CCCF)C1 ; C[C@H]1Cc2cc(OCCCC(F)(F)F)ccc2C=C1CN1CC(C(=O)Nc2ccncc2)C1 ; C[C@H]1Cc2cc(OCCCC(F)(F)F)ccc2C=C1CN1CC(C(=O)NCCC(F)(F)F)C1]
or a pharmaceutically acceptable salt or solvate thereof.

31. A pharmaceutical composition comprising a compound of any one of claims 1-30, or a pharmaceutically acceptable salt or solvate thereof, and at least one pharmaceutically acceptable excipient.

32. The pharmaceutical composition of claim 31, further comprising a peripherally restricted fatty acid amide hydrolase (FAAH) inhibitor.

33. The pharmaceutical composition of claim 32, wherein the peripherally restricted FAAH inhibitor is ASP-3652.

34. A method of treating a CNS disease or disorder in a patient in need thereof comprising administering to the patient a therapeutically effective amount of a compound of any one of claims 1-30, or a pharmaceutically acceptable salt or solvate thereof.

35. A method of treating a CNS disease or disorder in a patient in need thereof comprising administering to the patient a therapeutically effective amount of a pharmaceutical composition of any one of claims 31-33, or a pharmaceutically acceptable salt or solvate thereof.

36. The method of claim 34 or claim 35, wherein the CNS disease or disorder is selected from acute disseminated encephalomyelitis (ADEM), acute hemorrhagic leukoencephalitis (AHL or AHLE), adult Refsum disease, infantile Refsum disease, Alexander disease, Alzheimer's disease, Balo concentric sclerosis, Canavan disease, central pontine myelinolysis (CPM), cerebral palsy, cerebrotendineous xanthomatosis, chronic inflammatory demyelinating polyneuropathy (CIDP), Devic's syndrome, diffuse myelinoclastic sclerosis, encephalomyelitis, Guillain-Barre syndrome, idiopathic inflammatory demyelinating disease (HDD), Krabbe disease, Leber hereditary optic neuropathy, leukodystrophy, Marburg multiple sclerosis, Marchiafava-Bignami disease, metachromatic leukodystrophy (MLD), multifocal motor neuropathy (MMN), multiple sclerosis (MS), paraproteinemic demyelinating polyneuropathy, Pelizaeus-Merzbacher disease (PMD), progressive multifocal leukoencephalopathy (PML), tropical spastic paraparesis (TSP), X-linked adrenoleukodystrophy (X-ALD, ALO, or X-linked ALO), and Zellweger syndrome.

37. The method of claim 36, wherein the CNS disease or disorder is selected from multiple sclerosis and X-linked adrenoleukodystrophy.
